(19) United States
(12) Patent Application Publication
(10) Pub. No.: US 2023/0363913 A1
(43) Pub. Date: Nov. 16, 2023

(54) FASTENERS FOR PERCUTANEOUS DEVICES

(71) Applicant: Edwards Lifesciences Innovation (Israel) Ltd., Caesarea (IL)

(72) Inventors: Yaron Herman, Givat Ada (IL); Bezalel Haberman Browns, Jerusalem (IL); Eran Hoffer, Yahud (IL)

(21) Appl. No.: 18/354,872

(22) Filed: Jul. 19, 2023

Related U.S. Application Data
(63) Continuation of application No. PCT/IB2022/050127, filed on Jan. 9, 2022.
(60) Provisional application No. 63/140,226, filed on Jan. 21, 2021.

(51) Int. Cl. A61F 2/24

(52) U.S. Cl. CPC A61F 2/2466; A61F 2/2445; A61F 2230/0091

(57) ABSTRACT

An implant comprises a helical member. A guide assembly is used to guide the implantation of the helical member along a tissue of a heart. The guide assembly has a distal part that is transluminally advanceable to the heart, and comprises a guide frame that is intracardially expandable toward an expanded state, a guide rail, and multiple fasteners, configured to hold the guide rail in a guide arrangement around at least part of the guide frame. A driver of the system is configured to, while the guide rail is in the guide arrangement, anchor the helical member along an intracardial surface of tissue adjacent the guide frame, guided by the guide rail. Other implementations are also described.

### CROSS-REFERENCES TO RELATED APPLICATIONS

The present application is a continuation of International Patent Application PCT/IB2022/050127 to Herman et al., filed Jan. 9, 2022, which published as WO 2022/157592, and entitled “Fasteners for Percutaneous Devices,” which claims priority from Provisional U.S. Patent Application 63/140,226 to Herman et al., filed Jan. 21, 2021, and entitled “Fasteners for Percutaneous Devices,” each of which is incorporated by reference in its entirety for all purposes.

### BACKGROUND

Some percutaneous techniques, including transluminal techniques such as transcatheter cardiac interventions, require components to be decoupled from each other while inside the body of the subject being treated. For example, it may be advantageous for a tool that delivers and/or manipulates an implant to be reliably coupled to the implant until a certain time, and to then be reliably decoupled from the implant before the tool is withdrawn from the subject. In some instances, the components exert significant forces on the coupling prior to and during the decoupling. In some instances, it is advantageous that the mechanism of decoupling does not itself cause movement of the components that are being decoupled from each other.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

For some applications, fasteners for use with percutaneous devices including implants and delivery apparatuses or systems for such implants are described. The fasteners can comprise a longitudinal member, a bight of which can be formed into a loop that resists expansion and/or opening until it is intentionally released, and thereafter is easily expandable and/or openable.

The longitudinal member can comprise a braided and/or twisted material, such as a cord. A channel can extend transversally through the longitudinal member at a channel site, disposed between the two ends of the longitudinal member. In some applications in which the longitudinal member comprises a braided material, the longitudinal member may be threaded between strands of the braid, i.e., the channel is defined between strands of the braid. The loop is formed by a first end portion of the longitudinal member being threaded through the channel, such that the bight extends away from the first end portion and the channel, in a loop toward the second end of the longitudinal member and the channel site.

In order to inhibit enlargement and/or opening of the loop (i.e., to lock the fastener), a rod can extend transversally through the first end portion of the longitudinal member.

In some applications, the first end portion extends out of the channel (i.e., through and beyond the channel), away from the channel site and the bight of the loop. In some such applications, the rod extends transversally through the part of the first end portion that extends out of the channel, such that the rod inhibits enlargement of the loop by lying across an opening of the channel. For some applications, the first end portion loops back through the channel, and the rod extends through two sites of the first end portion.

For some applications, rather than the first end portion passing through a channel in the second end portion, the first end portion is looped around the second end portion. For some such applications, the rod extends through two sites of the first end portion, e.g., on either side of the loop.

For some applications, the rod can extend transversally through a part of the first end portion that is disposed within the channel, such that the rod inhibits enlargement of the loop by pinning the part of the first end portion to the walls of the channel. The fastener is unlockable by withdrawing the rod out of the first end portion of the longitudinal member.

In some applications, the fastener is used to constrain an implant in a crimped state, by arranging the loop of the fastener around the implant. The implant can be biased to assume an expanded state, such that without the fastener constraining the implant, the implant would self-expand. In some applications, the implant is delivered to the heart of a subject in its crimped state (e.g., within a catheter) while being constrained by the fastener, and once the implant is positioned within the heart, the implant is allowed to self-expand by withdrawing the rod from the longitudinal member, thereby allowing the loop to enlarge, or to open completely.

For some applications, a guide assembly is transluminally advanceable to a heart of the subject. The guide assembly comprises (i) a guide frame, deployable within the heart, (ii) one or more fasteners, secured to the guide frame, each of the fasteners defining a closed loop, and (iii) a guide rail. The guide rail may be fastened by (e.g., threaded through) the fasteners such that deployment of the guide frame at the site positions the guide rail along tissue. A tool is configured to position an implant along the tissue, guided by the guide rail, and to secure the implant to the tissue. This securing may be such that the implant becomes coupled to the guide frame by becoming disposed through the loops of the fasteners. The fasteners are unlockable within the heart, such that the guide frame becomes decouplable from the implant.

There is therefore provided, in accordance with some applications, a system and/or an apparatus including a fastener for use at a heart of a subject, the fastener including a rod, and a longitudinal member.

The longitudinal member can define (i) a first end portion, including a first end of the longitudinal member, (ii) a second end, (iii) a bight, and (iv) a channel site, disposed between the second end and the bight, and defining a channel that extends transversally through the longitudinal member.

The longitudinal member can be arranged such that the bight extends, away from the second end and the channel site, in a loop toward the first end portion, the first end portion extending from the bight and through the channel, thereby closing the loop.

The rod can extend transversally through the first end portion, such that the rod inhibits the first end portion from sliding through the channel.

In some applications, the longitudinal member is a first longitudinal member of a set of longitudinal members of the fastener.

In some applications, the set further includes a second longitudinal member defining: a first end portion, including a first end of the second longitudinal member, a second end, a bight, and a channel site, disposed between the second end of the second longitudinal member and the bight of the second longitudinal member.

In some applications, the channel site defines a channel that extends transversally through the second longitudinal member.

In some applications, the second longitudinal member is arranged such that the bight of the second longitudinal member extends, away from the second end of the second longitudinal member and the channel site of the second longitudinal member, in a loop toward the first end portion of the second longitudinal member, the first end portion of the second longitudinal member extending from the bight of the second longitudinal member and through the channel of the second longitudinal member, thereby closing the loop of the second longitudinal member.

In some applications, the rod further extends transversally through the first end portion of the second longitudinal member of the set, such that the rod inhibits the first end portion of the second longitudinal member from sliding through the channel of the second longitudinal member.

In some applications, the system and/or apparatus further includes an implant in a crimped state, and biased to assume an expanded state, wherein the longitudinal member is arranged around the implant such that the loop is tight around the implant, such that the rod's inhibition of the first end portion from sliding through the channel causes the longitudinal member to constrain the implant in the crimped state.

In some applications, the first end portion extends away from the bight and out of the channel, and the longitudinal member defines: a first opening of the channel, the first opening facing the bight, and a second opening of the channel, the second opening facing the first end, the rod extending transversally through the first end portion between the second opening and the first end, such that enlargement of the loop is limited by the rod abutting the second opening at the channel site.

In some applications, the rod extends transversally through the channel site and the first end portion in the channel.

In some applications, the fastener is configured such that the loop is openable by removing the rod from the first end portion and sliding the first end portion through the channel.

In some applications, the rod is rigid.

In some applications, the rod is flexible.

In some applications, the longitudinal member is longitudinally elastic.

In some applications, the longitudinal member includes a cord.

In some applications, the longitudinal member includes a polymer.

In some applications, the longitudinal member includes a suture.

In some applications, the loop defines a loop plane, and the rod extends transversally through the first end portion in an orientation that is substantially orthogonal to the loop plane.

In some applications, the loop defines a loop plane, and the rod extends transversally through the first end portion in an orientation that is substantially parallel with the loop plane.

In some applications, the rod has a length greater than 1 meter.

In some applications, the rod has a length that is less than 2 cm.

In some applications, the length of the rod is less than 1 cm.

In some applications, the length of the rod is less than 0.5 cm.

In some applications, the system and/or apparatus further includes a catheter defining a lumen, transluminally advanceable towards the heart of the subject, the longitudinal member being deliverable out of a distal end of the catheter.

In some applications, the rod is coupled to a tether, the tether being more flexible than the rod, and the fastener is deliverable out of the catheter while at least a part of the tether extends from the rod proximally through the catheter.

In some applications, the fastener is transluminally advanceable toward the heart of the subject such that bending of the catheter bends at least the part of the tether.

In some applications, the fastener is transluminally advanceable toward the heart of the subject while at least a part of the rod is disposed within the lumen of the catheter.

In some applications, the rod is flexible, and the fastener is transluminally advanceable toward the heart of the subject such that bending of the catheter bends at least the part of the rod.

In some applications, the system and/or apparatus further includes a deployable member, the deployable member coupled to the loop.

In some applications, at least part of the deployable member extends through the loop and is dimensioned with respect to the loop in a manner that inhibits sliding of the deployable member with respect to the loop.

In some applications, the loop is tight around the at least part of the deployable member.

In some applications, the deployable member defines a plurality of struts, and at least one strut of the plurality of struts extends through the loop.

In some applications, the deployable member is an implant.

In some applications, the deployable member is a fluoroscopic marker.

In some applications, the deployable member is a stent.

In some applications, the deployable member is a prosthetic heart valve.

In some applications, the deployable member is self-expanding.

In some applications, the deployable member is balloon-expandable.

In some applications, the fastener is configured such that the deployable member is decouplable from the loop by withdrawing the rod from the first end portion and sliding the first end portion through the channel.

In some applications, the system and/or apparatus further includes an elongate member, the fastener coupling the elongate member to the deployable member.

In some applications, the elongate member is fixed to the bight of the longitudinal member.

In some applications, the elongate member is configured to extend, from outside of the subject, transluminally to the heart of the subject, such that the deployable member is positionable within the heart of the subject by manipulating a proximal end of the elongate member.

In some applications, the system and/or apparatus further includes a stopper coupled to the elongate member, the stopper being wider than the elongate member such that the stopper inhibits sliding of the elongate member with respect to the loop.

There is further provided, in accordance with some applications, a method for use with an implant, the method including crimping the implant and, while the implant remains crimped, arranging a longitudinal member to form a loop around the implant such that a first end portion of the longitudinal member extends through a channel that extends transversally through the longitudinal member at a channel site of the longitudinal member.

In some applications, the method includes constraining the implant in a crimped state by inhibiting enlargement of the loop by piercing a rod through the first end portion, such that the rod inhibits the first end portion from sliding through the channel.

In some applications, piercing the rod through the first end portion includes piercing the rod transversally through the first end portion, such that enlargement of the loop is inhibited by the rod abutting the longitudinal member transversally across the channel site.

In some applications, the longitudinal member is a first longitudinal member of a set of longitudinal members, and the method further includes:
arranging a second longitudinal member of the set in a loop around the implant; and
piercing the rod through a first end portion of the second longitudinal member, such that the rod inhibits enlargement of the loop of the second longitudinal member.

In some applications, arranging the longitudinal member to form the loop around the implant includes arranging the longitudinal member to form the loop, and subsequently passing the loop over the implant.

In some applications, arranging the longitudinal member to form the loop around the implant includes forming the loop around the implant by inserting the first end portion through the channel site, and pulling the first end portion away from the channel site prior to piercing the rod through the first end portion.

In some applications, the longitudinal member is longitudinally elastic, arranging the longitudinal member to form the loop includes elastically stretching the longitudinal member by pulling the first end portion away from the channel site, piercing the rod through the first end portion includes, piercing the rod through the first end portion while the longitudinal member remains elastically stretched.

In some applications, the method further includes releasing the first end portion such that elastic contraction of the longitudinal member pulls the rod against the channel site.

In some applications, a part of the first end portion of the longitudinal member extends out of the channel and away from the channel site, and the rod extends transversally through the part of the first end portion, such that inhibiting enlargement of the loop includes inhibiting enlargement of the loop by the rod abutting the channel site.

In some applications, the rod extends transversally through the channel site and the first end portion in the channel.

In some applications, the method further includes transluminally advancing a catheter towards a heart of a subject, and, while the implant remains constrained in the crimped state by the longitudinal member, delivering the implant out of a distal end of the catheter.

In some applications, the method further includes facilitating expansion of the implant, by facilitating enlargement of the loop, by withdrawing the rod from the longitudinal member.

In some applications, the implant is biased to assume an expanded state, and facilitating expansion of the implant includes facilitating enlargement of the loop such that the implant self-expands.

In some applications, facilitating enlargement of the loop includes facilitating opening of the loop.

In some applications, the rod is flexible, delivering the implant out of the distal end of the catheter includes delivering the implant out of the distal end of the catheter such that the rod extends from the longitudinal member, proximally through the catheter and out of the subject, and withdrawing the rod from the longitudinal member includes withdrawing the rod from the longitudinal member by pulling the rod proximally from outside of the subject.

In some applications, the rod is coupled to a tether that is more flexible than the rod, delivering the implant out of the distal end of the catheter includes delivering the implant out of the distal end of the catheter such that the tether extends from the rod, proximally through the catheter and out of the subject, and withdrawing the rod from the longitudinal member includes withdrawing the rod from the longitudinal member by pulling the tether proximally from outside of the subject.

In some applications, delivering the implant out of the distal end of the catheter further includes delivering the rod entirely out of catheter, such that the tether extends into the distal end of the catheter and out of the subject.

The above method(s) and steps can be performed on a living animal or on a simulation, such as on a cadaver, cadaver heart, simulator (e.g., with the body parts, heart, tissue, etc. being simulated), etc.

There is further provided, in accordance with some applications, a system and/or an apparatus for use with tissue of a heart of a subject, the system and/or apparatus including a guide assembly, an implant, and a tool.

The guide assembly is transluminally advanceable to the heart, and can include: a guide frame, deployable at a site within the heart, one or more fasteners, secured to the guide frame, each of the fasteners defining a closed loop and being locked in a manner that maintains the loop, and a guide rail, threaded through the fasteners such that deployment of the guide frame at the site positions the guide rail along the tissue.

The tool can be configured to position the implant along the tissue, guided by the guide rail, and to secure the implant to the tissue in a manner in which the implant becomes coupled to the guide frame by becoming disposed through the loops of the fasteners.

The one or more fasteners can be unlockable within the heart, such that the guide frame becomes decouplable from the implant.

In some applications, the guide frame is configured to be deployed within an atrioventricular valve of the heart.

In some applications, the guide rail is held, by the fasteners, in an arc around at least part of the guide frame.

In some applications, the guide rail is radiopaque.

In some applications, the system and/or apparatus further includes a rod, and each one or more of the fasteners: includes a longitudinal member arranged to define the loop by an end portion of the longitudinal member extending through a channel that extends transversally through the longitudinal member, and is locked by the rod extending transversally through the end portion, and inhibiting sliding of the end portion through the channel.

In some applications, each of the one or more fasteners is unlockable by sliding the rod out of the end portion.

In some applications, the implant includes a helical member, implantable along the tissue by rotation of the helical member.

In some applications, the helical member has an axial length of 5-12 cm.

In some applications, the helical member has a sharpened tip.

In some applications, the helical member defines a central channel, and the implant is implantable along the tissue while the guide rail extends along the central channel.

In some applications, the guide rail is configured such that, while the implant remains implanted along the tissue with the guide rail extending along the central channel, the guide rail is axially slidable proximally through and out of the central channel.

In some applications, the system and/or apparatus further includes a contraction member, extending coaxially through a channel defined by the guide rail, the guide rail being axially slidable (i) proximally through and out of the central channel, and (ii) proximally over and along the contraction member, leaving the contraction member within the central channel.

In some applications, the system and/or apparatus further includes a stopper coupled to a distal end of the contraction member, such that tension applied to the contraction member longitudinally contracts the helical member, by the stopper inhibiting sliding of the contraction member through the central channel.

In some applications, the stopper is a first stopper, and the system and/or apparatus further includes a second stopper, configured to lock the tension in the contraction member by being coupled to the contraction member proximally from the helical member.

In some applications, the helical member defines a series of turns, and the implant is implantable along the tissue by screwing the helical member along a surface of the tissue such that a part each of the turns becomes embedded in the tissue and another part of each of the turns is disposed outside of the tissue.

In some applications, the guide rail is configured to limit a depth of penetration of the helical member into the tissue.

In some applications, the central channel has a diameter, and the guide rail has a thickness that is at least 25 percent of the diameter of the central channel.

In some applications, the thickness of the guide rail is at least 40 percent of the diameter of the central channel.

In some applications, the thickness of the guide rail is at least 50 percent of the diameter of the central channel.

In some applications, the thickness of the guide rail is at least 70 percent of the diameter of the central channel.

In some applications, the system and/or apparatus further includes a stopper coupled to a distal end of the guide rail, such that tension applied to the guide rail longitudinally contracts the helical member, by the stopper inhibiting the guide rail from sliding proximally through the central channel.

In some applications, the stopper is a first stopper, and the system and/or apparatus further includes a second stopper, configured to lock the tension in the guide rail by being coupled to the guide rail proximally from the helical member.

In some applications, the tissue is tissue of an annulus of a valve of the heart, and the implant is configured such that, after the implant has been implanted along the tissue, contraction of the helical member contracts the tissue of the annulus.

In some applications, the helical member is sufficiently flexible to follow the guide rail along the tissue.

In some applications, the helical member has a constant pitch.

There is further provided, in accordance with some applications, a method for use at a heart of a subject, the method including deploying a guide assembly at a site within the heart. The guide assembly includes a guide frame and fasteners. Each of the fasteners is secured to the guide frame and defines a closed loop.

In some applications, a guide rail is threaded through the loops for deploying the guide assembly at the site positions the guide rail along a tissue of the heart.

In some applications, the method includes implanting an implant along the tissue, guided by the guide rail, such that the implant becomes anchored to the tissue and disposed through the loops of the fasteners.

In some applications, the method includes, subsequently, within the heart, opening the loops and, while the implant remains implanted along the tissue, withdrawing the guide frame and the fasteners from the heart.

In some applications, the site is an atrioventricular valve of the heart, the tissue of the heart is tissue of an annulus of the atrioventricular valve, and deploying the guide assembly at the site includes deploying the guide frame within the atrioventricular valve, such that the guide rail becomes disposed along the annulus of the heart.

In some applications, the method further includes, subsequently to implanting the implant along the tissue, withdrawing the guide rail from the subject by sliding the guide rail out of the implant.

In some applications, at least a part of the guide assembly is radiopaque, and contracting the tissue includes contracting the tissue guided by at least one fluoroscopic image that includes the part of the guide assembly.

In some applications, each of the fasteners includes a longitudinal member, the guide assembly further includes at least one rod, and deploying the guide assembly includes deploying the guide assembly while each of the longitudinal members is: arranged to define the loop by an end portion of the longitudinal member extending through a channel that extends transversally through the longitudinal member and locked by the rod extending transversally through the end portion, and inhibiting sliding of the end portion through the channel.

In some applications, the method further includes, (i) subsequently to the implant becoming anchored to the tissue and disposed through the loops of the fasteners, and (ii) prior to opening the loops, unlocking the fasteners by removing the rod from the end portion.

In some applications, opening the loops includes opening the loops while the fasteners remain secured to the guide frame, and withdrawing the guide frame and the fasteners from the heart includes pulling the fasteners out of the heart by withdrawing the guide frame from the heart while the fasteners remain secured to the guide frame.

In some applications, the implant includes a helical member, and implanting the implant along the tissue includes screwing the helical member into the tissue via rotation of the helical member.

In some applications, withdrawing the guide frame and the fasteners from the heart includes withdrawing the guide frame and the fasteners from the heart while leaving at least part of the guide rail coupled to the helical member within the heart.

In some applications, screwing the helical member into the tissue via rotation of the helical member includes applying torque to a proximal end of the helical member.

In some applications, screwing the helical member into the tissue includes screwing the helical member into the tissue such that a screw axis of the helical member is disposed along a surface of the tissue.

In some applications, screwing the helical member into the tissue includes screwing the helical member into the tissue such that the helical member becomes at least partially embedded within the tissue.

In some applications, the helical member defines a plurality of turns, and screwing the helical member into the tissue includes screwing the helical member into the tissue such that a part of each of the turns becomes embedded in the tissue, and another part of each of the turns is disposed above the tissue.

In some applications, the helical member defines a central channel, and implanting the implant along the tissue includes implanting the helical member along the tissue while the helical member is threaded on the guide rail, with the guide rail extending through at least part of the central channel.

In some applications, implanting the helical member along the tissue includes implanting the helical member along the tissue while the guide rail limits a depth to which the helical member penetrates into the tissue.

In some applications, the method further includes, subsequently to withdrawing the guide frame and the fasteners from the heart: advancing a tensioning tool towards the heart; using the tensioning tool, contracting the tissue by applying tension to at least a part of the guide rail; and withdrawing the tensioning tool from the heart, such that at least the part of the guide rail and the helical member remain implanted along the tissue.

In some applications, the method further includes locking the tension in at least the part of the guide rail by locking a stopper to the guide rail.

The above method(s) and steps can be performed on a living animal or on a simulation, such as on a cadaver, cadaver heart, simulator (e.g., with the body parts, heart, tissue, etc. being simulated), etc.

There is further provided, in accordance with some applications, a method for use at a heart of a subject, the method including transluminally advancing a catheter towards the heart, the catheter coupled to a fastener and carrying a guide rail and delivering the guide rail out of the catheter and into the heart, such that the guide rail is threaded through a loop of the fastener and extends along a tissue of the heart. The method also includes, from the catheter, deploying an implant along the tissue, guided by sliding the loop over and along the guide rail.

In some applications, the method includes, subsequently, within the heart, decoupling the catheter from the guide rail by opening the loop.

In some applications, the tissue of the heart is tissue of an annulus of an atrioventricular valve of the heart, and delivering the guide rail includes delivering the guide rail out of the catheter and into the heart, such that the guide rail is threaded through the loop of the fastener and extends along the tissue of the annulus.

In some applications, the method further includes: subsequently to decoupling the catheter from the guide rail, transluminally advancing a tensioning tool toward the implant; subsequently, using the tensioning tool to apply tension to at least a part of the implant; and subsequently, withdrawing the tensioning tool from the subject.

In some applications, the guide rail includes a radiopaque material, and using the tensioning tool to apply the tension includes using the tensioning tool to apply the tension, facilitated by at least one fluoroscopic image that includes the guide rail.

In some applications, the guide rail includes a radiopaque material, and deploying the implant along the tissue includes deploying the implant along the tissue, further guided by at least one fluoroscopic image that includes the guide rail.

In some applications, the method further includes, prior to advancing the tensioning tool, withdrawing the catheter from the subject.

In some applications, advancing the catheter towards the heart includes advancing the catheter within a sheath towards the heart; withdrawing the catheter from the heart includes withdrawing the catheter through the sheath and out of the subject; and advancing the tensioning tool includes advancing the tensioning tool through the sheath towards the heart.

In some applications, the implant includes a contraction member, and applying tension to at least the part of the implant includes applying tension to the contraction member.

In some applications, the implant further includes a first tissue anchor and a second tissue anchor, and applying tension to the contraction member includes applying tension to the contraction member such that a distance between the first tissue anchor and the second tissue anchor becomes reduced.

In some applications, at least the second tissue anchor is slidably coupled to the contraction member, and applying tension to the contraction member includes sliding the contraction member with respect to at least the second tissue anchor.

In some applications, deploying the implant includes deploying the implant such that the first tissue anchor and the second tissue anchor are threaded onto the contraction member.

In some applications, the guide rail is defined by a guide frame; and delivering the guide rail includes deploying the guide frame within the heart such that the guide rail complements a shape of the tissue.

In some applications, the guide frame includes one or more struts projecting away from a plane defined by the guide frame, and deploying the guide frame within the heart includes delivering the guide frame within the heart such that each of the one or more struts projects away from the guide frame and extends through a respective commissure of an atrioventricular valve of the heart.

The above method(s) and steps can be performed on a living animal or on a simulation, such as on a cadaver, cadaver heart, simulator (e.g., with the body parts, heart, tissue, etc. being simulated), etc.

There is further provided, in accordance with some applications, a system and/or an apparatus for use with a tissue of a subject, the system and/or apparatus including a guide rail, an implant, and an implantation assembly. The implantation assembly can include, at a distal end portion thereof, a fastener that defines a loop, the implantation assembly being configured to transluminally place the guide rail along the tissue of the subject with the loop threaded on the guide rail.

In some applications, the implantation assembly is also configured to transluminally implant the implant along the tissue while being guided along the guide rail by the threading of the loop on the guide rail and intracorporeally decouple from the guide rail by opening the loop, such that the implantation assembly becomes withdrawable from the subject independently of the guide rail.

In some applications, the implantation assembly is transluminally advanceable towards the tissue of the subject with the loop of the fastener threaded on the guide rail.

In some applications, the guide rail includes a radiopaque material.

In some applications, the fastener includes a longitudinal member arranged to define the loop by a first end portion of the longitudinal member extending through a channel that extends transversally through the longitudinal member at a channel site of the longitudinal member, and a rod that extends transversally through the first end portion, and inhibiting opening of the loop by inhibiting sliding of the first end portion through the channel.

In some applications, the rod is withdrawable from the longitudinal member, withdrawal of the rod from the longitudinal member rendering the loop openable by sliding of the first end portion through the channel.

In some applications, the guide rail is compressible for transluminal delivery to the tissue and is intracorporeally expandable for positioning along the tissue.

In some applications, the guide rail has a predetermined expanded shape that complements a shape of the tissue, is compressible, from the predetermined expanded shape, into a compressed shape for transluminal delivery to the tissue and is intracorporeally expandable from the compressed shape toward the predetermined expanded shape.

In some applications, the tissue includes tissue of a native heart valve annulus.

In some applications, the guide rail is shaped to define: (1) a base frame having a shape such that it tracks a circumference of the native heart valve annulus, and (2) one or more struts projecting away from a plane defined by the base frame, the one or more struts providing an indicator of one or more commissures of a native heart valve.

In some applications, the implant includes at least one tissue anchor.

In some applications, the implant includes at least two tissue anchors.

In some applications, the implant further includes a contraction member, slidably coupled to the at least two tissue anchors.

In some applications, the at least two tissue anchors are threaded onto the contraction member.

In some applications, the system and/or apparatus further includes a tensioning tool, adapted to apply tension to the contraction member.

There is further provided, in accordance with some applications, a method for use at a heart of a subject, the method including transluminally advancing, towards the heart, an implant coupled to a fastener that includes: a longitudinal member arranged in a loop such that a first end portion of the longitudinal member extends through a channel that extends transversally through the longitudinal member at a channel site of the longitudinal member and a rod that extends transversally through the first end portion, and inhibits sliding of the first end portion through the channel.

In some applications, the method includes, while the implant remains coupled to the fastener, delivering the implant out of a catheter and into the heart and, subsequently, withdrawing the rod from the first end portion.

In some applications, transluminally advancing, toward the heart, the implant coupled to the fastener includes transluminally advancing, toward the heart, at least part of the implant extending through the loop of the fastener.

In some applications, transluminally advancing, toward the heart, the implant coupled to the fastener includes transluminally advancing, toward the heart, the implant coupled, by the fastener, to an elongate member, for positioning the implant within the heart; and prior to withdrawing the rod from the first end portion, using the elongate member to position the implant within the heart.

In some applications, withdrawing the rod from the first end portion includes withdrawing the rod from the first end portion such that the implant is released from the loop, and the fastener remains coupled to the elongate member.

The above method(s) and steps can be performed on a living animal or on a simulation, such as on a cadaver, cadaver heart, simulator (e.g., with the body parts, heart, tissue, etc. being simulated), etc.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

FIGS. 1-4 are schematic illustrations of a fastener and variants thereof, in accordance with some applications;

FIGS. 5-7 are schematic illustration of systems and techniques for uses of fasteners as a component of a system for positioning an implant, in accordance with respective applications;

FIGS. 8A-C are schematic illustrations of systems and techniques for arranging a fastener, in accordance with some applications;

FIGS. 9A-I and 10 are schematic illustration of systems and techniques for uses of multiple fasteners within systems for guiding the implantation of an implant, in accordance with respective applications;

FIG. 11 and FIGS. 12A-G are schematic illustration of systems and techniques for uses of a fastener as a component of a system for guiding the implantation of an implant, in accordance with respective applications; and

FIGS. 13A-B, 14, and 15 are schematic illustrations of fasteners and variants thereof, in accordance with some applications.

### DETAILED DESCRIPTION

Reference is made to FIGS. 1-5, 13A-B, and 14, which are schematic illustrations of examples of a fastener 40 (e.g., variants 40a, 40b, 40c, and 40d thereof), and techniques for use therewith, in accordance with some applications. Fastener 40 comprises a longitudinal member 20, arranged in a loop 28. FIG. 1 shows longitudinal member 20 and includes an inset that illustrates that the longitudinal member can comprise a braided material, such as a cord. However, it is to be noted that, for some applications, the longitudinal member can comprise a different material and/or have a different structure. For example, longitudinal member 20 can comprise one or more of a string, a ribbon, a polymer, a protein, and a metal. Longitudinal member 20 can comprise a suture.

Longitudinal member 20 has a first end portion 20a that includes a first end 21 of the longitudinal member, a second end portion 20c that includes a second end 22 of the longitudinal member, and a bight 20b between the first end portion and the second end portion. Between first end 21 and second end 22, longitudinal member 20 has a channel site 26 at which the longitudinal member defines a channel 24, extending transversally through the longitudinal member. That is, channel 24 extends transversally through the body of longitudinal member 20 at channel site 26. In some applications in which longitudinal member 20 comprises a braided material, the longitudinal member may be threaded between strands of the braid, i.e., channel 24 is defined between strands of the braid.

Longitudinal member 20 have a thickness that is at least 0.1 mm (e.g., at least 0.2 mm, e.g., at least 0.3 mm) and/or no more than 1 mm (e.g., no more than 0.5 mm, e.g., no more than 0.4 mm, such as no more than 0.3 mm). For example, longitudinal member 20 can have a thickness of 0.2-0.4 mm. For some applications, longitudinal member 20 can be a suture of USP designation 2-0, 3-0, or 4-0.

Loop 28 is formed by first end portion 20a being threaded through channel 24. In the resulting arrangement of longitudinal member 20, bight 20b extends away from first end portion 20a and channel 24, and loops back toward channel site 26. FIG. 4 illustrates an example of how this can be achieved, by looping a bight of a thread 80 around first end portion 20a, while the two free ends of the thread are disposed on the other side of channel 24 (i.e., such that two lengths of the thread extend through the channel), and then pulling first end portion 20a through the channel by pulling on the ends of the thread.

In order to inhibit enlargement and/or opening of loop 28 (e.g., to maintain the longitudinal member in a loop), a rod 30 extends transversally through the first end portion 20a, thereby inhibiting (e.g., barring) the first end portion from slipping through channel 24 (i.e., from slipping through the channel in a direction that would enlarge and/or open the loop). That is, the presence of rod 30 through first end portion 20a locks fastener 40. Although rod 30 can be sufficiently long and flexible for transluminal use (e.g., as described hereinbelow), it can be sufficiently rigid to not flex and slip through channel 24. For example, rod 30 can comprise a wire (e.g., comprising a metal such as Nitinol or stainless steel, and/or comprising a polymer). However, it is to be noted that, for some applications, the rod can comprise a different material and/or have a different structure. In some applications, an end of the rod defines a sharp piercing tip, for piercing through first end portion 20a. In some such applications, the piercing tip comprises a strong and inflexible material.

Rod 30 can have a thickness greater than 0.01 mm and/or less than 1 mm (e.g., 0.05-0.5 mm, e.g., 0.05-0.2 mm, such as 0.1 mm).

Rod 30 can have a length greater than 0.1 cm (e.g., 0.1-2 cm, e.g., 0.1-1 cm, e.g., 0.2-0.5 cm, such as 0.5 cm) and/or less than 2 m (e.g., 1-2 m, such as 1.5 m).

It is hypothesized that the small size of fastener 40 is thereby advantageous for its use in percutaneous (e.g., transluminal) techniques, such as (but not limited to) those described hereinbelow. It is further hypothesized that the simplicity of fastener 40, including the absence of catches, pawls, detents, or other protrusion-like components, and of complex machine-like mechanisms, advantageously confers increased reliability on the fastener, especially when the fastener is experiencing significant forces (e.g., is under stress) when it is required to open. For example, it is hypothesized that even when longitudinal member 20 is under significant tension, fastener 40 remains reliably unlockable because rod 30 remains reliably slidable out of channel 24.

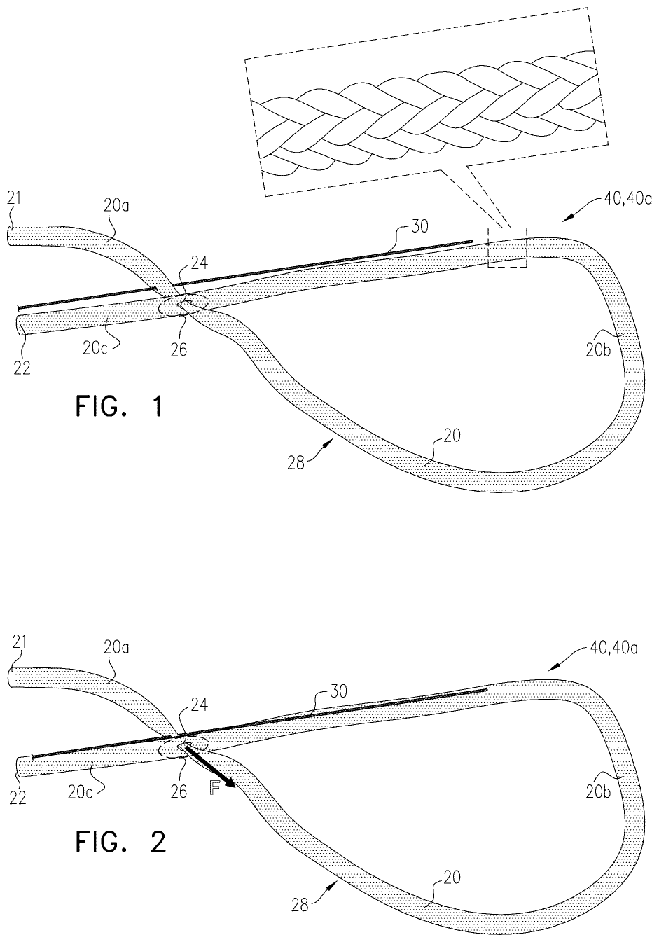

FIGS. 1-2 illustrates a fastener 40a, which is a variant of fastener 40, in which rod 30 extends transversally through a part of first end portion 20a that extends out of channel 24, such that pulling the first end portion towards channel site 26 and bight 20b pulls the rod against an opening of the channel, such that the rod lies against the opening, effectively barring the rod, and thus the first end portion, from slipping through the channel and enlarging loop 28. In some applications, in order to maximally inhibit slipping of first end portion 20a through channel 24, rod 30 extends through the first end portion at close proximity to channel site 26.

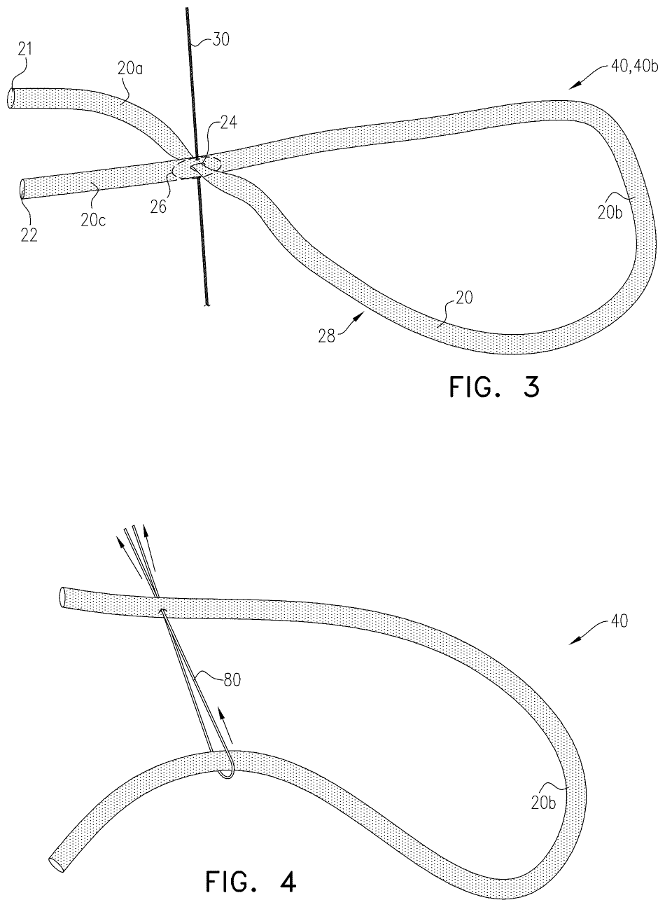

FIG. 3 illustrates a fastener 40b, which is a variant of fastener 40, in which rod 30 extends transversally through a part of first end portion 20a that is disposed within channel 24, such that the rod inhibits enlargement of loop 28 by pinning the part of the first end portion to the walls of the channel.

Loop 28 can be considered to lie on (e.g., to define) a loop plane. For some applications, rod 30 extends transversally through first end portion 20a in an orientation that is substantially parallel with, or is even coincident with, the loop plane (e.g., as shown in FIGS. 1-2). For some applications, rod 30 extends transversally through first end portion 20a in an orientation that is substantially orthogonal to the loop plane (e.g., as shown in FIG. 3).

Loops 28 can be releasable by withdrawing rod 30 from first end portion 20a, such that the first end portion is allowed to slip through channel 24 in a manner that enlarges and/or opens the loop. For some applications, rod 30 is long and flexible, such that it can be delivered transluminally, with (e.g., within) a catheter, through the vasculature of the subject, towards the heart. Thus, fasteners 40 can be releasable by withdrawing the rod from the longitudinal members by pulling the rod proximally, from outside the subject.

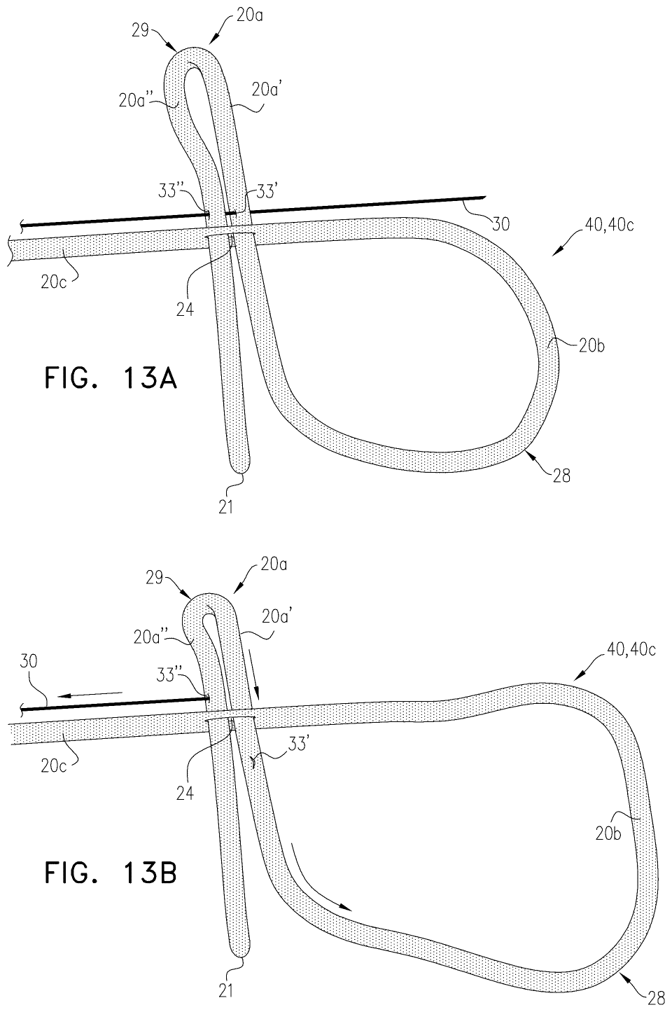

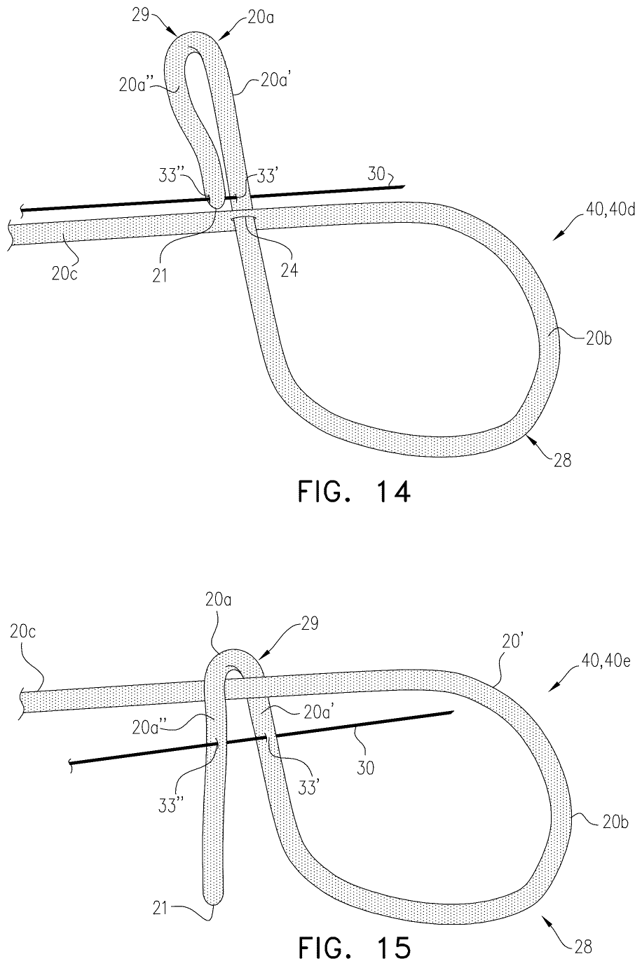

FIGS. 13A-B and 14 show fasteners 40c and 40d respectively, which are variants of fastener 40. In contrast to the examples described hereinabove in which rod 30 extends through first end portion 20a at only a single site, FIGS. 13A-14 illustrate examples in which the rod extends through the first end portion at both a first site 33′ and a second site 33″, such that the first end portion is secured in a secondary loop 29. Secondary loop 29 can be disposed between the first site and the second site.

First site 33′ is disposed at a first part 20a′ of the first end portion, and second site 33″ is disposed at a second part 20a″ of first end portion 20a. First part 20a′ extends, from primary loop 28 and the channel, towards secondary loop 29. Second part 20a″ extends, from secondary loop 29, back towards channel 24 and first end 21. In some applications, fasteners 40c and/or 40d may be formed by first forming secondary loop 29, and then pushing the secondary loop through channel 24.

Fasteners 40c and 40d are configured such that retraction of rod 30 proximally by a first distance causes the rod to exit from first site 33′ but not from second site 33″, such that the first site but not the second site becomes slidable through channel 24. It is hypothesized that this may advantageously allow for a preliminary expansion of loop 28 without fully opening the loop (i.e., without fully releasing the fastener). This is illustrated in the transition between FIG. 13A to FIG. 13B, which shows loop 28 expanding slightly upon retraction of the rod from the first site, and first site 33′ having slid through channel 24. In applications in which fastener 40c is used with an implant (e.g., as described hereinbelow), it is hypothesized that this may provide an operator with the opportunity of withdrawing the rod from first site 33′, and allowing loop 28 to enlarge slightly (e.g., to loosen), and to then determine whether to complete the implantation of the implant. Should it be determined that implantation is suboptimal, fastener 40c may allow repositioning and/or retrieval of the implant because the loop remains closed by rod 30 remaining extended through second site 33″.

It is hypothesized that, for some applications, forces applied by rod 30 to first portion 20a may be mitigated by secondary loop 29 and/or by the rod extending through the first portion at two sites. For example, a force that may pull the first end portion towards channel 24 may be distributed more broadly across the first end portion. Similarly, the presence of rod 30 at second site 33″ may reduce the chance of the first end portion fraying in response to the rod pressing against first site 33′ as the rod resists such forces from pulling the first portion through channel 24. This may furthermore advantageously allow first end portion 20a to require less excess length in order to prevent this unwanted fraying.

In fastener 40c, second part 20a″ extends back through channel 24 toward first end 21.

In fastener 40d, second part 20a″ does not extend back through the channel, such that the longitudinal member only extends through the channel at first part 20a′. In such an example, it is hypothesized that withdrawing rod 30 from only first site 33′ would lead to an intermediate arrangement that is similar to that shown for fastener 40a, in which rod 30 only extends through first portion 20a at a single site.

Reference is now made to FIG. 15, which illustrates a fastener 40e, in accordance with some applications. Fastener 40e may be considered to be a variant of fastener 40.

Fastener 40e comprises a longitudinal member 20′ which may be identical to longitudinal member 20 described hereinabove, except that longitudinal member 20′ may not define a channel therethrough and is not threaded through itself. Rather, first portion 20a is formed into secondary loop 29 by the first portion looping around second portion 20c. Similarly to fasteners 40c and 40d, secondary loop 29 is secured by rod 30 extending through first portion 20a at first site 33′ and second site 33″, the secondary loop being between the first site and the second site.

Also similarly to fasteners 40c and 40d, first site 33′ is disposed at first part 20a′ of the first end portion, and second site 33″ is disposed at a second part 20a″ of first end portion 20a. First part 20a′ extends, from primary loop 28 towards secondary loop 29. Second part 20a″ extends, from secondary loop 29, back towards first end 21. It is to be noted that first part 20a′, second part 20a″, and rod 30 collectively define a closed loop around second portion 20c. That is, secondary loop 29 and rod 30 collectively define a closed loop around second portion 20c.

It is hypothesized that, for some applications, the arrangement shown for fastener 40e may advantageously obviate the requirement for a channel to be defined through the longitudinal member. It is further hypothesized that, for some applications, this arrangement may provide similar advantages to those described for fasteners 40c and 40d, e.g., that withdrawing the rod from the first site would allow for a slight enlargement of the primary loop without releasing the fastener.

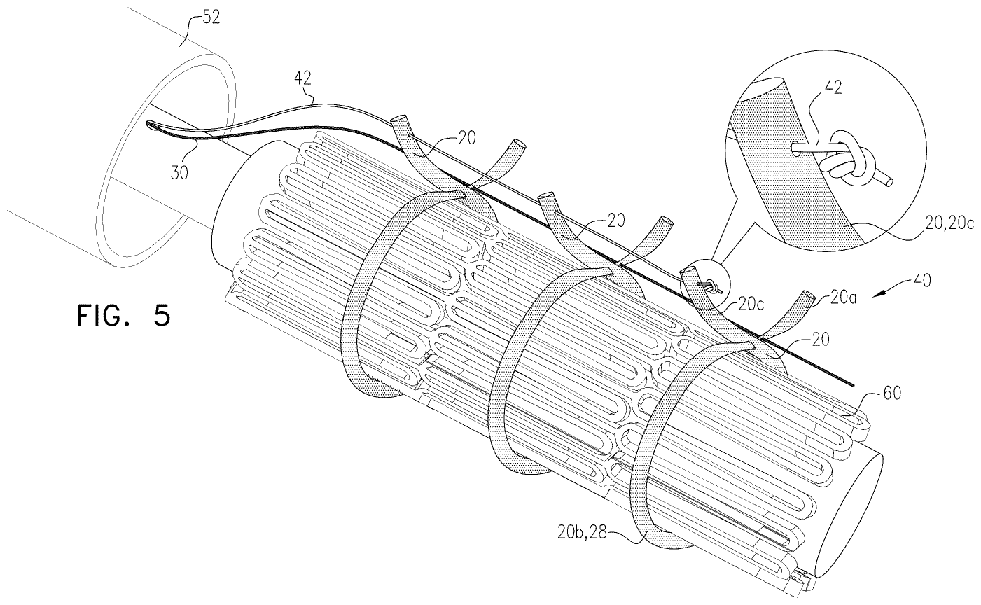

In some applications a single rod 30 can be used to lock multiple fasteners (e.g., to inhibit the enlargement and/or opening of a plurality of loops 28), by extending through first end portions 20a of the loops. FIG. 5 illustrates such an application, in which multiple fasteners 40 are shown being used to constrain, in a crimped state, an implant 60 such as a prosthetic heart valve or a stent. In some applications, implant 60 can be biased to assume an expanded state (i.e., is self-expanding), such that loops 28 constrain the implant from self-expanding. The implant can be deliverable transluminally, optionally via a catheter 52, while constrained in its crimped state by the fastener. In some applications, prior to constraining implant 60 with fasteners 40, implant 60 is crimped tightly, and loops 28 are then arranged around the crimped implant. For some applications, fasteners 40 can be pre-assembled, and loops 28 passed over the crimped implant.

It is to be noted that, although implant 60 can initially expand slightly until it presses against loops 28, its state as constrained by fasteners 40 can still be considered to be its crimped state. Once implant 60 is positioned within the heart (e.g., has been delivered out of a distal end of catheter 52), rod 30 can be withdrawn from loops 28. Implant 60 can be biased to assume the expanded state once freed of the constraint of the loops, such that the implant self-expands within the heart. That is, withdrawing rod 30 from first end portions 20a facilitates (e.g., triggers) expansion and/or opening of the loops, such that the implant is allowed to self-expand, and thus become deployed, within the heart. It is hypothesized that, despite the expansive forces applied by implant 60 on loop 28, rod 30 remains slidable out of channel 24 without excessive pulling of the rod that might otherwise inadvertently move implant 60.

For some applications, implant 60 can be balloon-expandable.

As described hereinabove, rod 30 can be sufficiently long and flexible for transluminal use. For example, during transcatheter use of fastener 40 (e.g., when a catheter such as catheter 52 is used to deliver an implant such as implant 60), the fastener can be transluminally advanceable toward the heart while at least part of the rod is disposed within the lumen of the catheter. For example, rod 30 can easily bend in response to bending of the catheter through which it extends.

It is to be noted that, for some applications, an arrangement of multiple longitudinal members 20 restrained (e.g., locked) as loops 28 by a single rod 30, such as the arrangement shown in FIG. 5, can optionally be considered to be a single fastener that comprises multiple longitudinal members and a single rod.

A connector 42 can be used to connect longitudinal members 20, such that once implant 60 has been deployed within the heart, by withdrawing rod 30, the longitudinal members are retractable from the heart by withdrawing the connector, through the catheter and out of the subject.

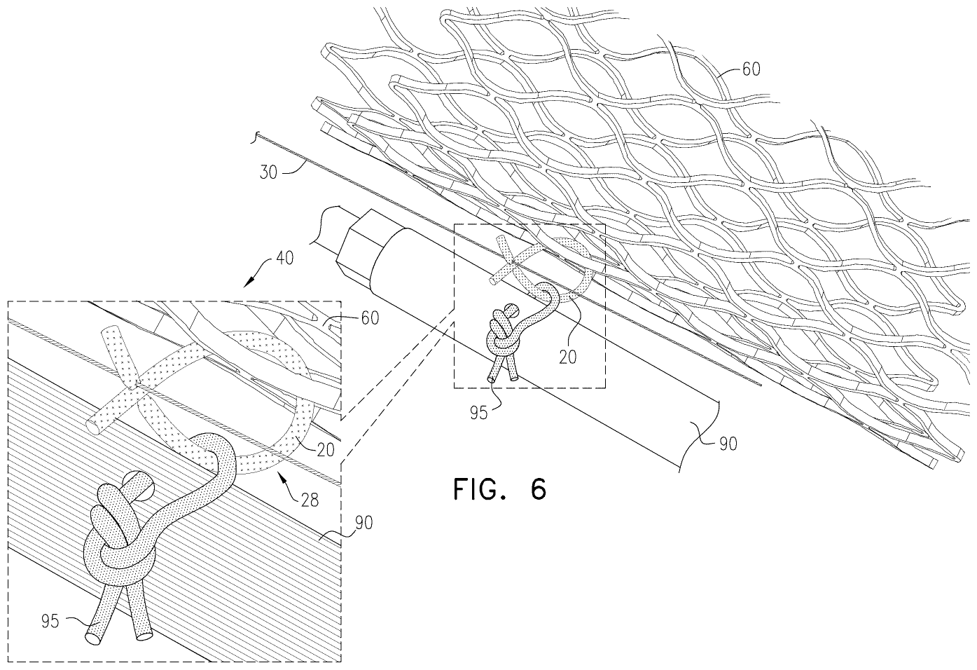

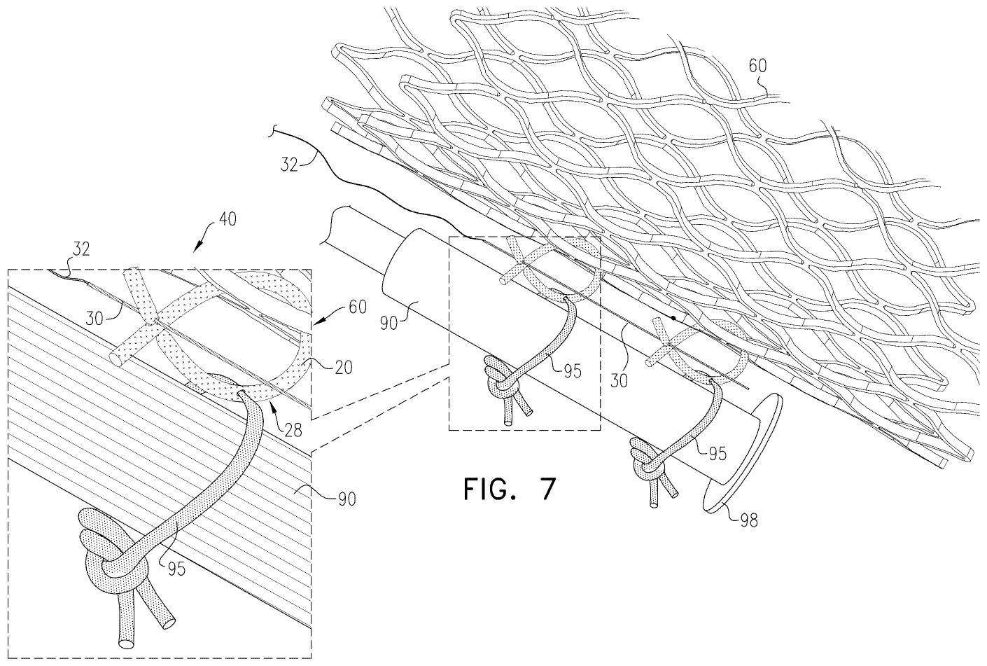

Reference is now made to FIGS. 6 and 7, which are schematic illustrations of applications in which implant 60 is positionable within the heart using a positioning tool 90 (e.g., comprising an elongate member, such as a shaft) that is coupled to the implant using at least one fastener 40. It is to be noted that another deployable member can be used in place of implant 60, mutatis mutandis.

Positioning tool 90 can be long and at least partially flexible, such that, once the implant is disposed within the heart, the positioning tool can extend, from the heart where it is coupled to the implant, proximally through the vasculature and out of the subject, such that the positioning tool, and thus the implant, are manipulatable by adjusting the positioning tool extracorporeally.

Coupling of positioning tool 90 to implant 60 by fastener 40 can be achieved by the fastener being fixed to the positioning tool, and loop 28 being threaded onto/around at least part of implant 60. For some applications, and as shown, implant 60 comprises multiple struts, e.g., arranged in a cellular structure. For some such applications, and as shown, loop 28 is threaded around a strut of implant 60 (e.g., through one or more cells of the cellular structure of the implant), thereby securely coupling positioning tool 90 to the implant.

For some applications, positioning tool 90 is fixed to longitudinal member 20, such that once the fastener is released (i.e., by withdrawing rod 30), the longitudinal member remains secured to the positioning tool. For example, a securing element 95, secured to positioning tool 90, can extend through loop 28 (e.g., as shown in FIG. 6), or through a transverse hole in bight 20b or second end portion 20c of longitudinal member 20 (e.g., as shown in FIG. 7). Examples of how securing element 95 can be secured to positioning tool 90 include a closed loop, defined by the securing element, extending around the positioning tool (e.g., as shown in FIG. 7), and/or through a passageway defined through the positioning tool (e.g., as shown in FIG. 6). The securing element can be configured in a variety of ways, e.g., as a suture, line, wire, clip, clasp, etc.

For some applications, a stopper 98 is coupled to a distal end of tool 90 and is wider than at least the distal end of the tool. Stopper 98 inhibits loop 28 from sliding with respect to (e.g., distally off of) tool 90.

Implant 60 can be positioned and repositioned using tool 90, e.g., prior to and/or after deployment of the implant at the implantation site. Once a desired position has been achieved, implant positioning tool 90 can then be decoupled from the tool by retracting rod 30, such that loop 28 can open. As described elsewhere herein, mutatis mutandis, the ease of sliding rod 30 out of channel 24 is hypothesized to facilitate opening of fastener 40 without inadvertently moving implant 60.

As described hereinabove, rod 30 can be sufficiently long and flexible to extend transluminally to the heart of the subject. However, for some applications, rod 30 can be short (e.g., having a length of 1-10 mm, e.g., 2-8 mm, such as 2-5 mm), and attached to a tether 32 that can extend from the rod in the heart, transluminally out of the subject (e.g., as shown in FIG. 7). For example, during transcatheter use of fastener 40, the fastener can be deliverable out of the catheter while at least part of the tether extends from the rod, proximally through the catheter, e.g., such that rod 30 is withdrawable from longitudinal member 20 by pulling the tether proximally from outside of the subject. Tether 32 can be more flexible than rod 30 (e.g., the rod can be rigid), and this flexibility can further facilitate transcatheter techniques. For example, the tether may easily bend in response to bending of the catheter through which it extends. Although the use of tether 32 is shown only in FIG. 7, it is to be understood that it is applicable to the other applications described herein, mutatis mutandis.

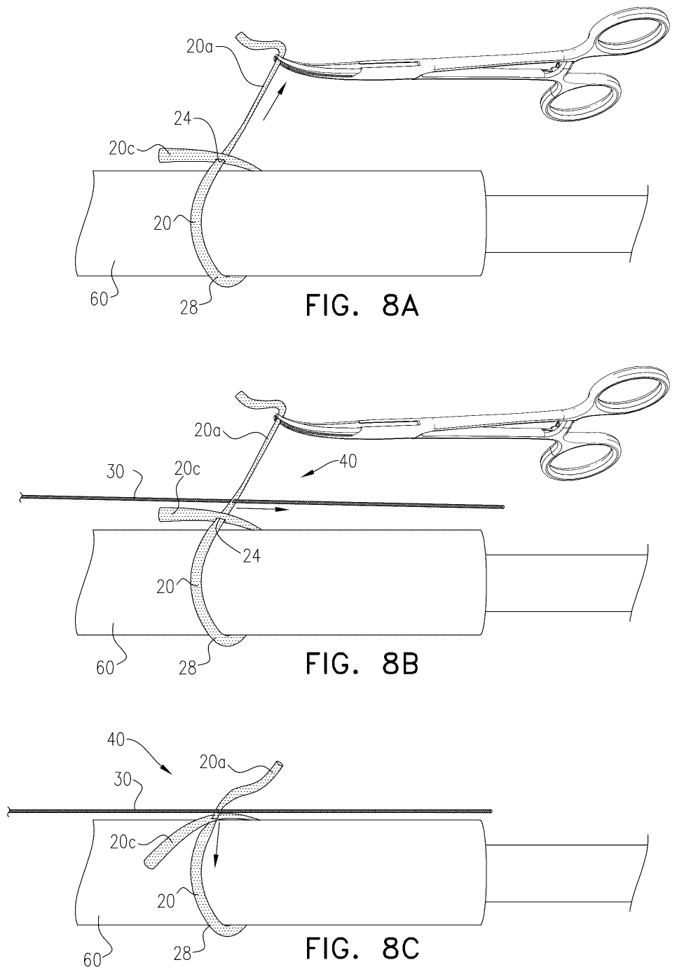

Reference is made to FIGS. 8A-C, which are schematic illustrations of an application in which longitudinal member 20 is longitudinally elastic, in accordance with some applications. In some applications, it is desirable that fastener 40 be arranged such that loop 28 is tight against the item (e.g., implant 60) therewithin, e.g., so as to firmly grip the item, e.g., to prevent sliding of the item with respect to the loop. It is hypothesized that configuring applications of longitudinal member 20 to be elastic may facilitate achieving such tightness. In some such applications, first end portion 20a is pulled through and away from channel 24 to an extent sufficient to stretch longitudinal member 20 (FIG. 8A). Rod 30 is subsequently inserted through first end portion 20a, often close to channel 24 (FIG. 8B). Subsequently, first end portion 20a is released, such that elastic contraction of the longitudinal member pulls rod 30 against channel site 26 and squeezes loop 28 against the implant (FIG. 8C).

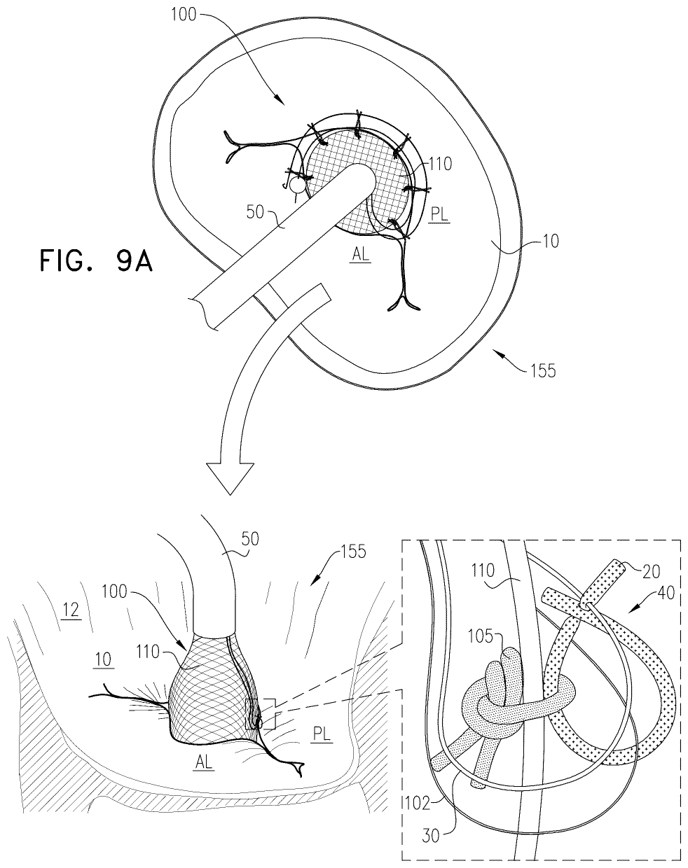

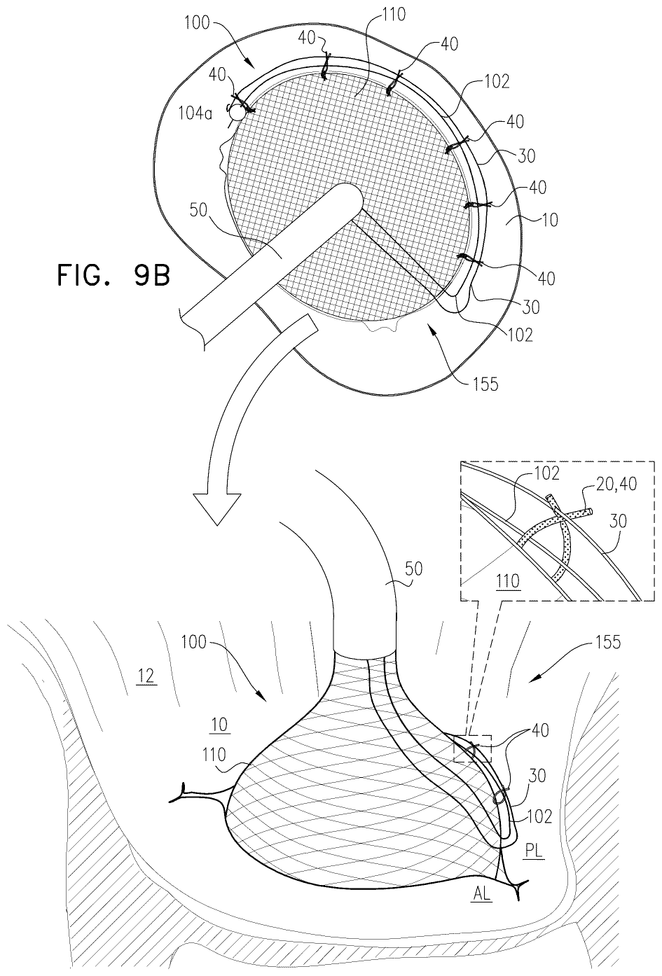

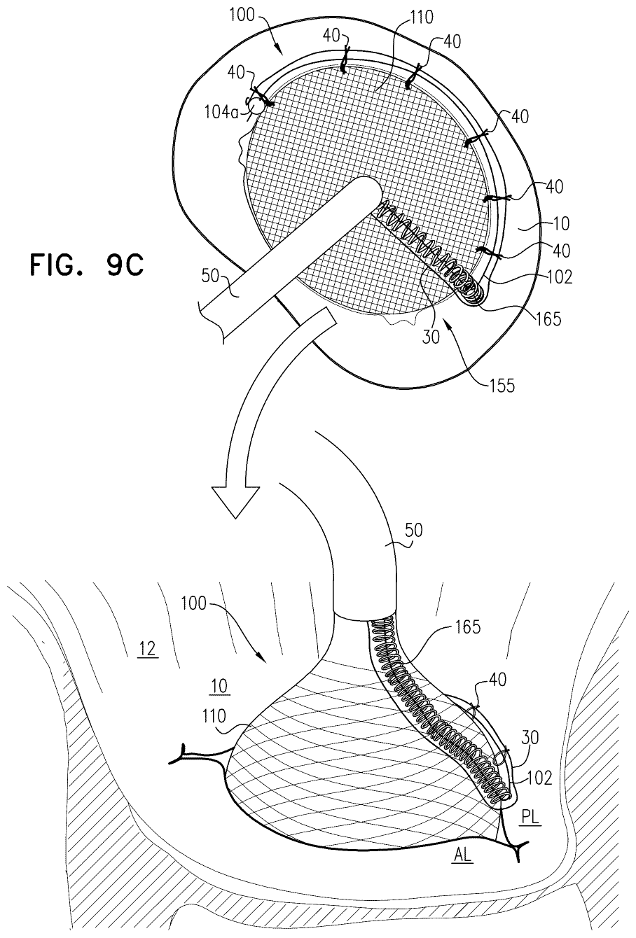

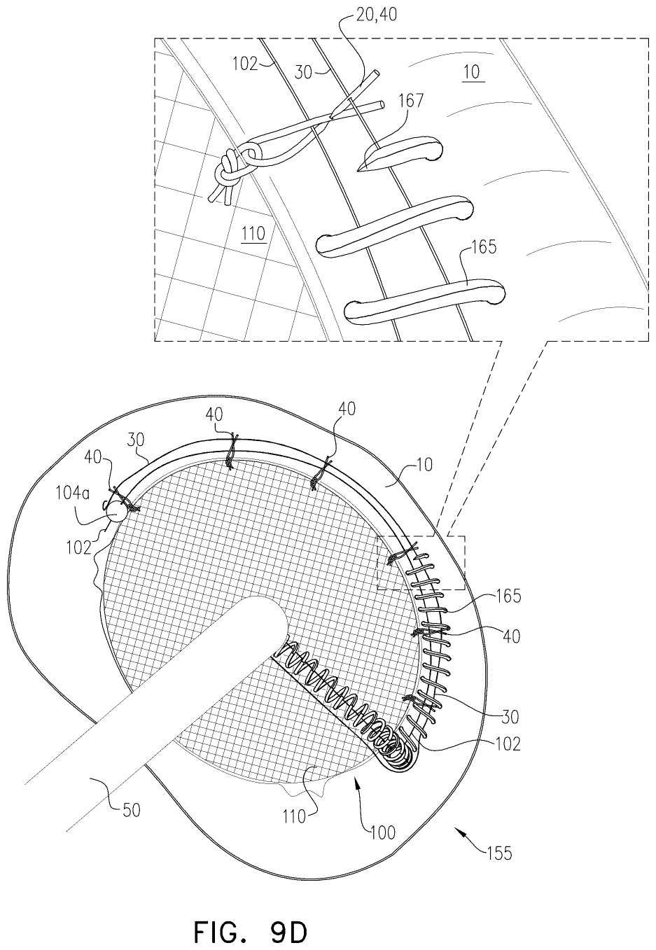

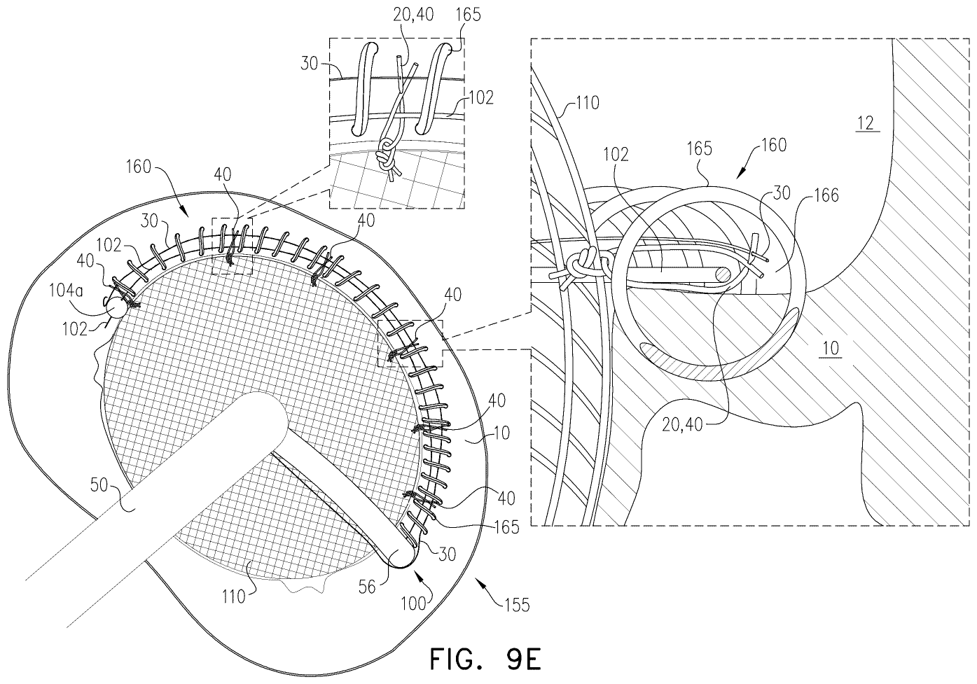

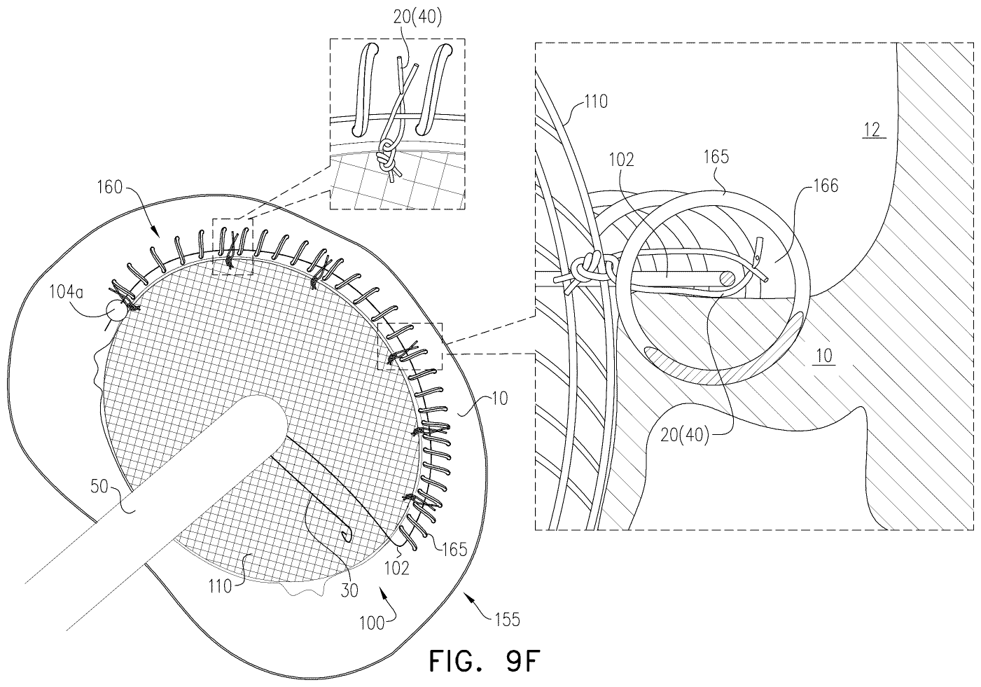

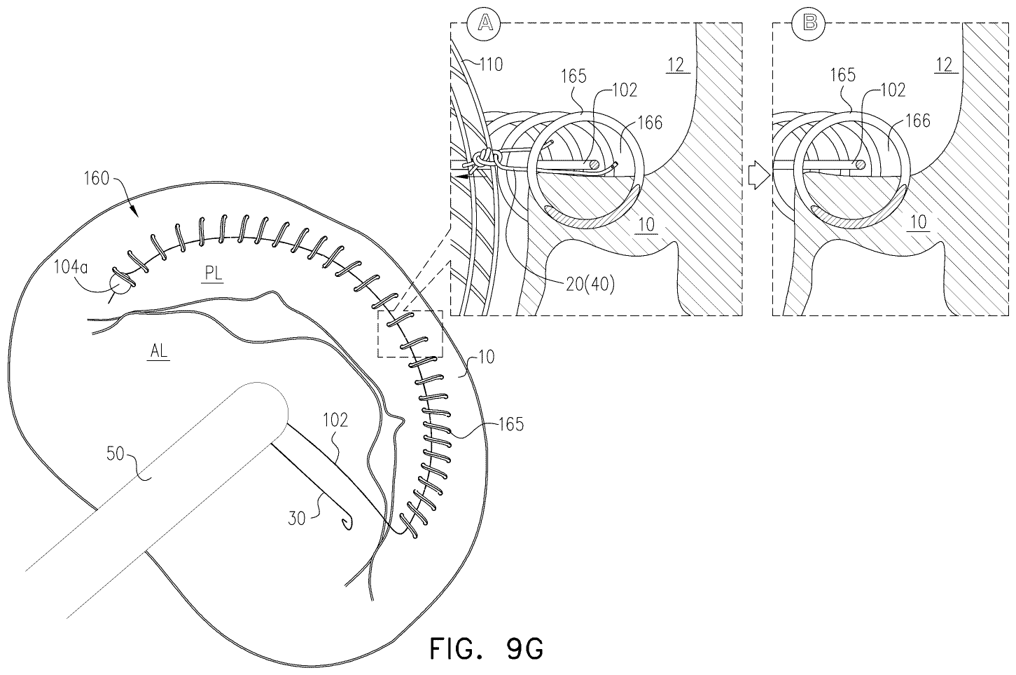

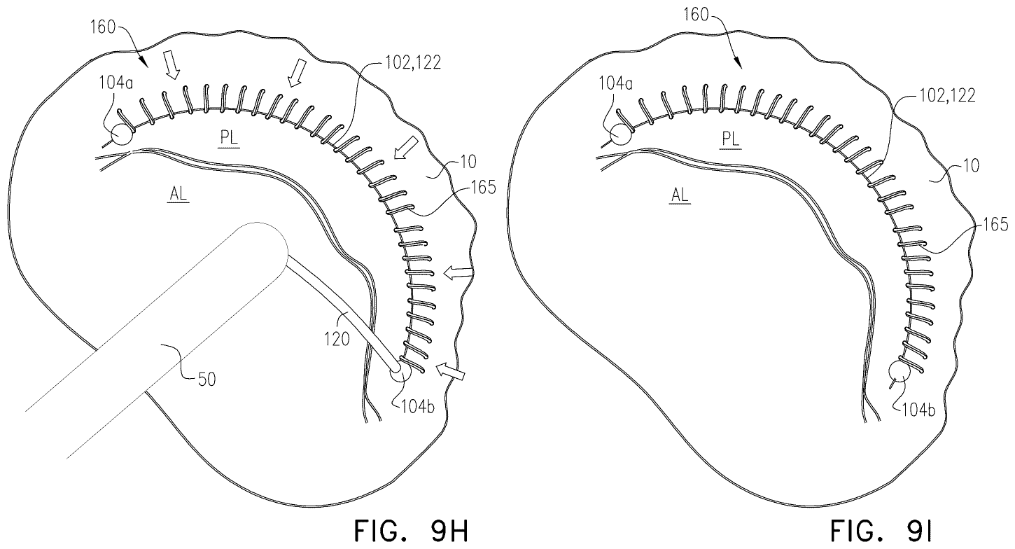

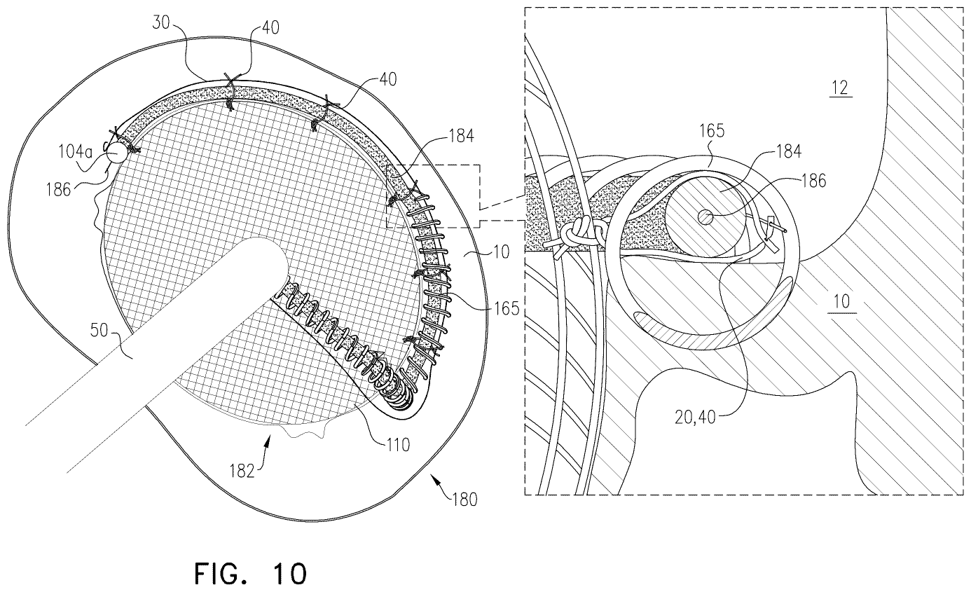

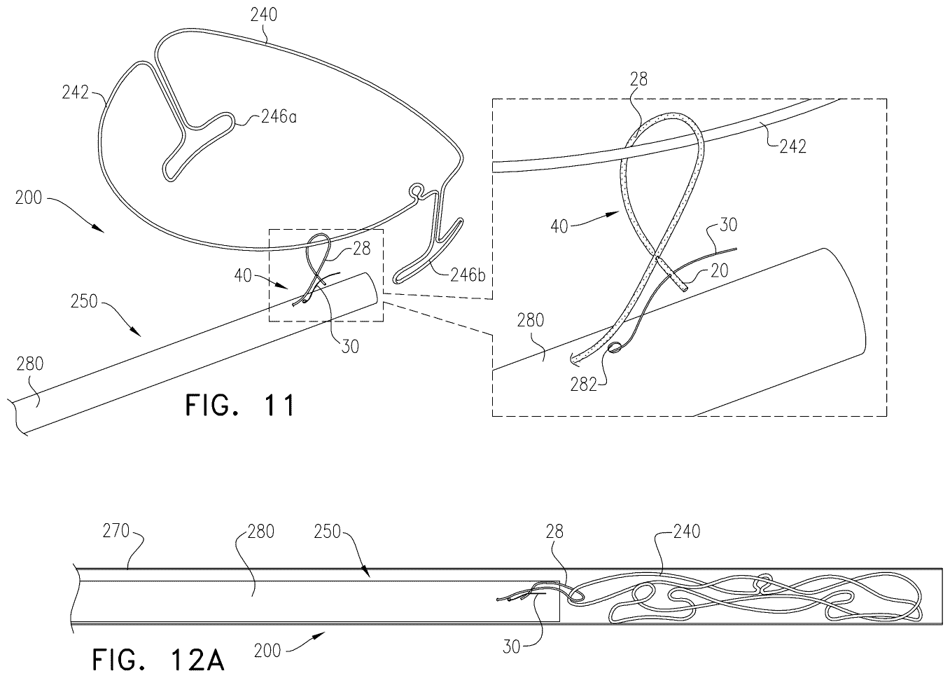

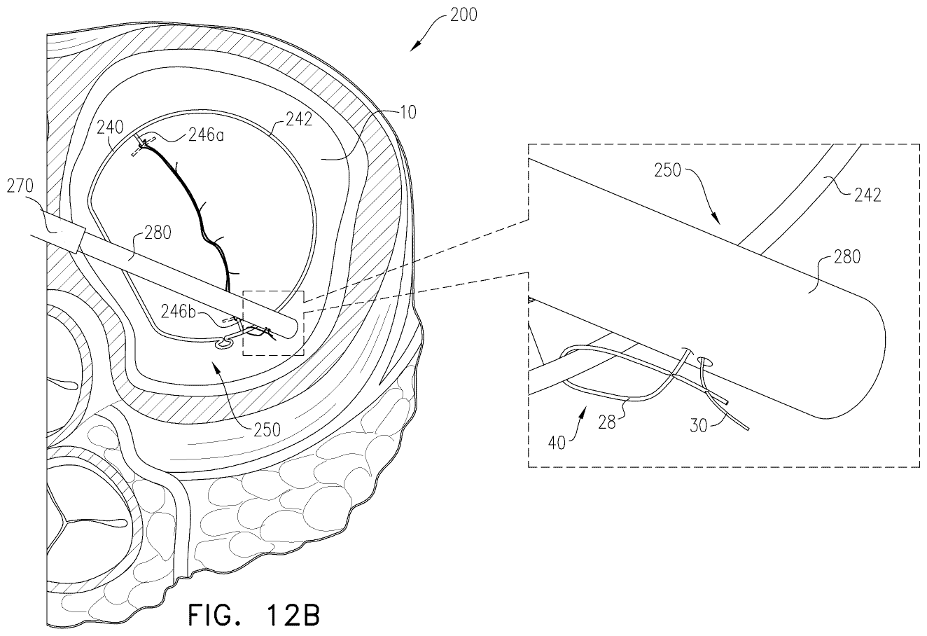

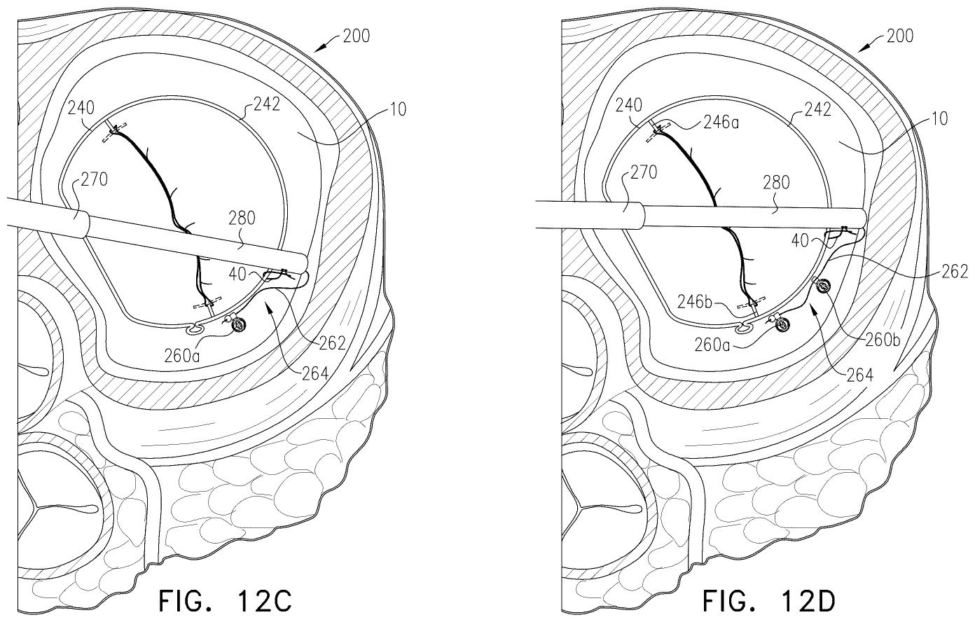

Reference is now made to FIGS. 9A-I, which are schematic illustrations of a use of fasteners 40 as a component of a system 155, in accordance with some applications. System 155 comprises a guide assembly 100 and an implant 160, and can be for use at an atrioventricular valve of a heart of a subject.

Guide assembly 100 comprises a guide rail 102 for guiding the implantation of implant 160 along the tissue of a heart valve, e.g., an annulus 10 of the heart valve (e.g., as part of an annuloplasty procedure), in accordance with some applications.

Guide assembly 100 often also comprises a guide frame 110, expandable (e.g., by being self-expanding, or by being balloon-expandable) within the heart of the subject. For some applications, and as shown, guide frame 110 is expanded within an atrioventricular valve, e.g., the atrioventricular valve that is being treated. Expansion of the guide frame pushes at least part of leaflets AL and PL radially outward, and often results in the guide frame pressing against the tissue of the annulus (e.g., pushing leaflets AL and PL down and away from the annulus). Nonetheless, the valve often continues to function at least in part, e.g., because guide frame 110 is open and allows blood flow therethrough, and/or because leaflets AL and PL remain partially functional (e.g., downstream of the guide frame), providing a net one-way flow of blood through the valve.

Multiple fasteners 40 are fixed along a part of the circumference of guide frame 110, with the longitudinal members of the fasteners constrained (e.g., by rod 30 extending through first end portions 20a) into loops 28, with guide rail 102 threaded therethrough, e.g., such the guide rail extends circumferentially around at least part of the guide frame. Thus, positioning guide frame 110 within the valve positions guide rail 102 along the tissue of the annulus (e.g., in contact with an atrial surface of the annulus, or slightly upstream of the atrial surface). As described hereinbelow, guide rail 102 is to serve as a guide for implantation of implant 160, e.g., defining, at least in part, a shape that implant 160 will assume upon its implantation.

Guide assembly 100 can be advanced transluminally (e.g., transfemorally), via a catheter 50, while guide frame 110 is in a contracted state, and, once deployed out of a distal end of the catheter, guide frame 110 expands within the heart (FIG. 9A), positioning guide rail 102 along the tissue of annulus 10 (FIG. 9B). For some applications, and as shown, guide rail 102 further extends, from guide frame 110, proximally away from the heart. For some applications, rod 30 also extends proximally away from guide frame 110 and the heart (e.g., the rod extends proximally through catheter 50 and out of the subject, such that the rod is withdrawable from fasteners 40 while the fasteners are disposed within the heart).

In some applications, implant 160 comprises a helical member 165, defining a helix, the helix extending around a central channel 166 defined by the helix. For some applications, the helical member is adapted to be anchored into tissue (e.g., of annulus 10) via rotation (i.e., screwed into the tissue). For some applications, helical member 165 has a sharpened tip 167, adapted to facilitate the anchoring of the helical member into the tissue. Helical member 165 can be sufficiently flexible to be transluminally advanced to the heart, and to follow guide rail 102. However, helical member 165 can be also sufficiently rigid that it can be screwed into the tissue, often by applying torque to a proximal end of the helical member. For example, helical member 165 may generally exhibit deflectional flexibility (i.e., its central longitudinal axis may be easily deflected), but may generally not exhibit torsional flexibility (e.g., the helix may not be easily untwisted (i.e., unwound) or further twisted). For some applications, in its resting state, helical member 165 has an axial length (i.e., a length along its central axis, rather than a helical length along its helical shape) of 2-20 cm (e.g., 2-12 cm, e.g., 3-12 cm, such as 5-12 cm).

For some applications, helical member 165 is configured to have a constant pitch along its length, and/or is configured such that the pitch remains generally constant during anchoring to the tissue. Nonetheless, for some applications, and as described hereinbelow, helical member 165 can be axially contracted (i.e., reducing its pitch) subsequent to its anchoring to the tissue.

For some applications, once guide assembly 100 is in place, helical member 165 is advanced out of catheter 50, and along guide rail 102, e.g., with the guide rail threaded through central channel 166 (FIGS. 9C-E). Implant 160 is implanted along the tissue, guided by guide rail 102, such that the guide rail directs the implantation of the implant along the tissue. That is, guide rail 102 extends along the tissue such that it provides a track along which the implant progresses. Guide rail 102 can thereby define the shape that implant 160 will assume upon implantation. As shown, guide rail 102 can arc around at least part of guide frame 110, and helical member 165 can thereby be anchored in an arc around at least part of the annulus.

For some applications, guide rail 102 can extend along the tissue (e.g., of annulus 10) in a manner that complements (e.g., generally matches) the shape of the tissue. For some such applications, this can be facilitated by guide frame 110 being sufficiently compliant that its expanded shape is influenced by the existing shape of the tissue.

Helical member 165 can be implanted along the tissue such that it becomes at least partially screwed into the tissue. For example, an anchor driver 56 can be coupled to a proximal part of helical member 165 and can drive the helical member into the tissue by rotation of the helical member. Guide rail 102 often directs this such that a part of each turn of helical member 165 becomes embedded in tissue of annulus 10, and another part of each turn is disposed outside of the tissue (e.g., in atrium 12). For some applications, a screw axis of helical member 165 is disposed along the surface of the tissue. For some applications, the screw axis can be parallel with the surface of the tissue, but within the tissue (e.g., the helical member is deeper in the tissue), or parallel with the surface of the tissue but within the atrium (e.g., the helical member is shallower in the tissue). FIGS. 9C-E schematically illustrate an application in which the implant becomes partially embedded within the tissue during implantation, such that part of each turn of the helical member becomes submerged in the tissue, and part of each turn remains above the tissue.

In some applications, subsequently to anchoring helical member 165 along the tissue, at least part of guide assembly 100 is withdrawn from the heart, often via catheter 50. For example, and as shown, guide frame 110, with fasteners 40 still attached thereto, can be withdrawn.

For some applications, during implantation, guide frame 110 becomes coupled to tissue of annulus 10 via implant 160, and it becomes necessary to decouple the guide frame from the implant in order to remove the guide frame from the subject. For example, and as described hereinbelow with reference to FIGS. 9H-I, in some applications, a distal portion of guide rail 102 additionally serves as (e.g., becomes) a contraction member 122 of implant 160, e.g., at least a distal portion of the guide rail can remain in the heart chronically, as a component of the implant. In some such applications, because helical member 165 anchors guide rail 102 to the tissue, in order to facilitate the withdrawal of guide frame 110 from the heart independently of the guide rail, fasteners 40 are opened, thereby releasing the guide frame from the guide rail. This can be achieved by withdrawing rod 30 from longitudinal members 20 (FIG. 9F), such that fasteners 40 become allowed to open, e.g., in response to being pulled, by guide frame 110, away from the guide rail (e.g., as schematically illustrated by the transition between the insets of FIGS. 9F and 9G). In FIG. 9F, reference numeral 40 is shown in parentheses because longitudinal members 20 are no longer constrained by rod 30, and therefore no longer function as fasteners.

It is hypothesized that the opening of fasteners 40 described hereinabove can also advantageously free the fasteners (and thereby guide frame 110 coupled thereto) should helical member 165 have become threaded through loop 28 of one or more of the fasteners during screwing of the helical member along the tissue.

In some applications, once helical member 165 has been anchored along the tissue, adjustment (e.g., contraction) of the annulus is performed by applying tension to guide rail 102, such that a distal portion of the guide rail serves as (e.g., becomes) a contraction member 122 of implant 160 (FIG. 9H). For example, at least in part due to a first stopper 104a that is fixed to a distal end of guide rail 102, thereby preventing the distal end of the guide rail from sliding proximally through helical member 165, tensioning of guide rail 102 (i.e., contraction member 122) results in longitudinal contraction of the helical member. A tensioning tool 120, delivered out of catheter 50, can be used to facilitate application of the tension.

Once the tension has been applied, the tension is locked by affixing a second stopper 104b to guide rail 102, often proximally from helical member 165, e.g., such that contraction member 122 is defined as the portion of guide rail 102 disposed between first stopper 104a and second stopper 104b. Stopper 104b can be applied by tool 120. For some applications, the tension is applied by pulling on the guide rail 102 while simultaneously applying an opposing force via tensioning tool 120, e.g., by pushing second stopper 104b distally. FIG. 9I shows implant 160 implanted along annulus 10, once tension has been applied to guide rail 102 (i.e., to contraction member 122).

It is to be noted that although FIGS. 9A-I show guide assembly 100 being used at the heart of a subject, e.g., for guiding an annuloplasty procedure, the guide assembly can be used with any tissue, and for other medical procedures.

For some applications, the guide rail is withdrawn from the subject after the helical member has been at least partially implanted along the tissue. For example, the guide rail may have no stopper fixed to its distal end and may be pulled proximally through and out of the central channel of the helical member. For such applications, the guide rail is therefore not (e.g., does not become) a component of the implant.

Reference is made to FIG. 10, which is a schematic illustration of a system 180 for use at a valve of a heart of a subject, in accordance with some applications. System 180 can be identical to system 155, except for as noted. System 180 comprises a guide assembly 182 that comprises a guide rail 184 for guiding the implantation of helical member 165 along the tissue of an annulus 10 of a native heart valve (e.g., as part of an annuloplasty procedure), in accordance with some applications. Guide assembly 182 often also comprises a guide frame such as guide frame 110.

Like guide rail 102, described hereinabove, guide rail 184 serves to guide anchoring of helical member 165, but guide rail 184 can be thicker than guide rail 102, and can also be more rigid. Furthermore, guide rail 184 often has no stopper fixed to its distal end and can be configured to be pulled proximally through and out of central channel 166 of helical member 165 after the helical member has been at least partially implanted along the tissue.

The additional thickness of guide rail 184 may limit the depth to which helical member 165 can penetrate into the tissue, e.g., by the guide rail abutting the surface of the tissue. For example, guide rail 184 can have a thickness that is at least 25 percent (e.g., at least 40 percent, e.g., at least 50 percent, e.g., at least 70 percent) of the diameter of central channel 166 of helical member 165 (i.e., the internal diameter of the helical member). In order to facilitate penetration of helical member 165 into the tissue, and/or withdrawal of guide rail 184 from the helical member, the thickness of the guide rail can be no more than 95 percent (e.g., no more than 90 percent, e.g., no more than 80 percent, such as no more than 70 percent) of the diameter of central channel 166.

As described hereinabove, guide rail 184 can be configured to be removed after helical member 165 has been anchored. Therefore, guide rail 184 does not (e.g., cannot) serve as a contraction member. For some applications, a separate contraction member (e.g., a contraction wire) 186 is provided, which extends through central channel 166 of helical member 165, e.g., coaxially with guide rail 184 (such as through a lumen defined by the guide rail, as shown), or alongside the guide rail, and which remains behind after the guide rail is withdrawn. For some applications, a contraction member can be introduced after helical member 165 has been implanted. For some applications, no distinct contraction member is used, e.g., helical member 165 itself adjusts the tissue.

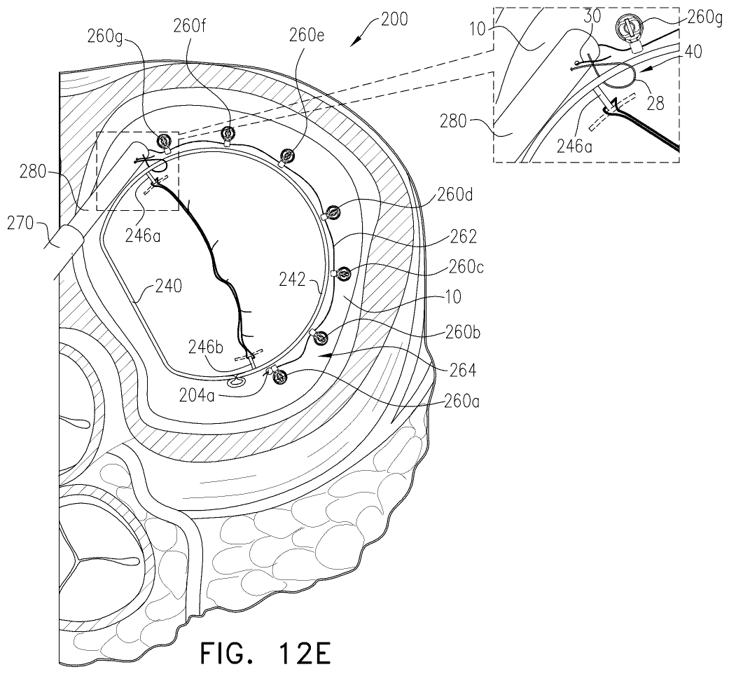

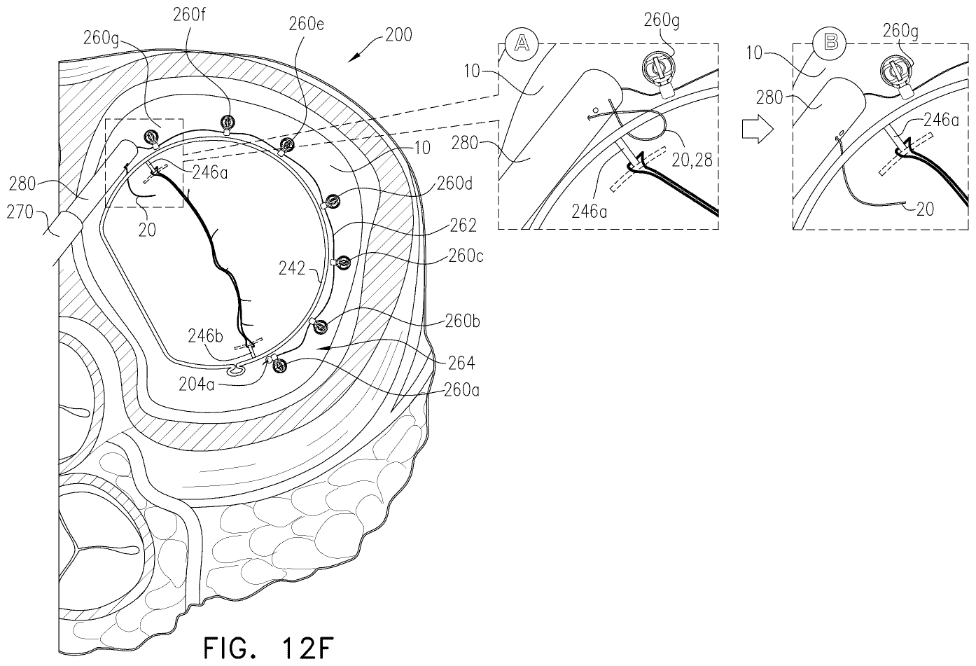

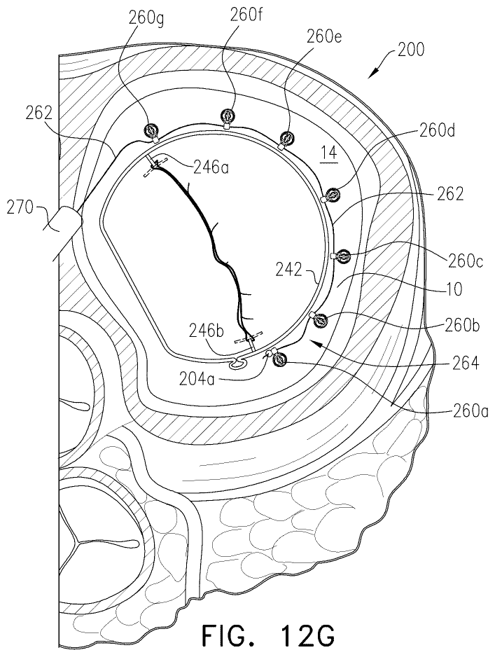

Reference is now made to FIGS. 11 and 12A-G which are schematic illustrations of a use of fastener 40 as a component of a system 200, in accordance with some applications. System 200 comprises an implant 264, a guide rail 242, and an implantation assembly 250 that is configured to implant the implant guided by the guide rail.

System 200 often comprises a base frame 240, adapted to be fitted at annulus 10 such that a part of the base frame defines guide rail 242, extending along an upstream side of the annulus, where implant 264 is to be implanted. For example, and as shown, base frame 240 can define a ring (which can be circular or noncircular), part of which defines guide rail 242. As schematically illustrated in FIG. 11, base frame 240 can define one or more (e.g., two or three) struts 246 (e.g., a strut 246a, and a strut 246b), projecting away from a plane defined by the base frame, configured to extend downstream through respective commissures of the valve. Struts 246 support and/or stabilize base frame 240 at the valve. Thus, positioning base frame 240 at the valve positions guide rail 242 along the tissue of the annulus (e.g., in contact with an atrial surface of the annulus, or slightly upstream of the atrial surface). As described hereinbelow, guide rail 242 is to serve as a guide for implantation of implant 264, e.g., defining, at least in part, a shape that implant 264 will assume upon its implantation.

Base frame 240 (e.g., guide rail 242 thereof) can further provide a fluoroscopic indicator of the anatomy, further facilitating guided implantation of implant 264.

Implantation assembly 250 comprises a tool 280 (e.g., comprising a catheter, a shaft, and/or a driver), adapted to be transluminally (e.g., transfemorally) advanced, to the heart of the subject, often within a sheath 270. Tool 280 can define an inner lumen, and implant 264 can be deliverable via inner lumen and out of a distal end of the tool. Base frame 240 can be coupled to a distal end-portion of tool 280 via fastener 40 and remains so once base frame 240 is deployed at the valve. Tool 280 can be long and at least partially flexible, such that, while base frame 240 is disposed at the valve, the tool can extend, from the base frame, proximally through the vasculature and out of the subject.

FIG. 12A shows a delivery state of implantation assembly 250, in which the implantation assembly is deliverable transluminally toward the heart, within sheath 270, while base frame 240 is coupled to tool 280. In the delivery state, base frame 240 can be compressed within sheath 270. For some applications, and as shown, fastener 40 is disposed laterally from (e.g., is coupled to) an outer surface of the distal end-portion of tool 280. For some applications, rod 30 is disposed laterally from the distal end-portion of tool 280, e.g., by extending out of a lateral opening 282 in the tool. For example, rod 30 can extend along tool 280 within a secondary lumen of the tool, exiting the secondary lumen at opening 282.

Base frame 240 can be configured to, upon deployment, automatically expand toward a predetermined expanded shape that complements a shape of the tissue.

In some applications, implant 264 comprises a plurality of tissue anchors 260a-g, adapted to be anchored to (e.g., screwed into) tissue of annulus 10 (e.g., such that an anchoring/screw axis of the tissue anchor is generally orthogonal to the surface of the tissue). Tissue anchors 260a-g can be connected to each other via a contraction member (e.g., a tether) 262, which can be slidably coupled to the tissue anchors (e.g., by being threaded through an eyelet defined by the head of each tissue anchor), such that once the tissue anchors have been implanted along the tissue, applying tension to contraction member 262 draws the anchors together, thereby reducing the circumference of the annulus.

For some applications, once implantation assembly 250 is in place, e.g., with guide rail 242 threaded through loop 28 of tool 280 (FIG. 12B), the tool is positioned at a first site along the guide rail for the implantation of first tissue anchor 260a. First tissue anchor 260a is then anchored to the tissue at the first site, facilitated by tool 280 (e.g., is deployed out of the distal end of the tool). As described hereinabove, tool 280 can comprise a driver—this driver can drive the tissue anchor into the tissue.

Subsequently, a second site is selected along the tissue for the implantation of a second tissue anchor 260b, by advancing tool 280 along guide rail 242, via the sliding of loop 28 of fastener 40 over the guide rail, such that it becomes positioned at the second site (FIG. 12C). A second anchor 260b is anchored to the tissue at the second site. This process is repeated (FIGS. 12D-E), with tool 280 sliding along guide rail 242, such that the guide rail provides a track along which the implant is implanted. Guide rail 242 can thereby define the shape that implant 264 will assume upon implantation. As shown, tissue anchors 260 can be implanted along the tissue such that contraction member 262 forms an arc around at least part of the annulus.

It is to be noted that the number of tissue anchors 260 shown is illustrative, and implant 264 can comprise more or fewer tissue anchors.

In some applications, once implant 264 has been deployed (e.g., implanted) along the tissue (e.g., once tissue anchors 260a-g have been anchored along the tissue), tool 280 is decoupled from guide rail 242 by releasing fastener 40, e.g., by withdrawing rod 30 from longitudinal member 20, such as by withdrawing the rod into tool 280 (FIG. 12F). Tool 280 is subsequently withdrawn from the heart, leaving implant 264, and often also base frame 240, within the heart (FIG. 12G).

In some applications, adjustment (e.g., contraction) of the annulus is subsequently performed by applying tension to contraction member 262, thereby reducing the distance between tissue anchors 260, e.g., such that the contraction member slides with respect to the tissue anchors. For example, a tensioning tool, which can be similar or identical to tensioning tool 120, delivered via sheath 270, can be used to facilitate application and locking of the tension, e.g., as described with reference to FIGS. 9H-I, mutatis mutandis. For some applications, such a tensioning tool (e.g., tool 120) is not advanceable through sheath 270 in the presence of tool 280, and it is hypothesized that it therefore advantageous for fastener 40 to facilitate intracorporeal decoupling of tool 280 from guide rail 242, and base frame 240 as a whole.

In some applications, at least a portion of base frame 240 (e.g., at least a portion of guide rail 242) comprises a radiopaque material, which can further facilitate implantation and/or adjustment of implant 264. For example, mechanical guidance of tool 280 by guide rail 242 can be augmented by fluoroscopic guidance, e.g., guided by at least one fluoroscopic image that includes the guide rail. For example, fluoroscopy can facilitate identification of a location of a distal end of the tool with respect to the guide rail, e.g., a position of the distal end along the guide rail. Similarly, fluoroscopy can be used to ensure that the distal end of the tool is disposed radially outward from the guide rail (i.e., further than the guide rail from the leaflets of the valve). Furthermore, base frame 240 can be configured to contract during contraction of annulus 10, thereby facilitating fluoroscopic monitoring of annulus 10 during tensioning of contraction member 262. It is hypothesized that, at least for some applications, it is therefore advantageous to retain base frame 240 in place during adjustment of implant 264, and that it is therefore advantageous for fastener 40 to facilitate intracorporeal decoupling of tool 280 from the base frame.

Systems, apparatuses, devices, implants, methods, and techniques described hereinabove can be used, mutatis mutandis, in combination with systems, apparatuses, devices, implants, methods, and/or techniques disclosed in U.S. Provisional Patent Application 62/877,785 to Sheps et al., filed Jul. 23, 2019, and entitled “Fluoroscopic visualization of heart valve anatomy,” and/or PCT application PCT/IL2020/050807 filed Jul. 22, 2020, and entitled “Fluoroscopic visualization of heart valve anatomy,” each of which is incorporated herein by reference for all purposes. Further, the techniques, methods, steps, etc. described or suggested herein or in these incorporated references can be performed on a living animal or on a non-living simulation, such as on a cadaver, cadaver heart, simulator (e.g., with the body parts, tissue, etc. being simulated), etc.

It is to be understood that throughout the application, reference to fastener 40 may be referring to any of its variants, e.g., fasteners 40a, 40b, 40c, 40d, and 40e—or combinations thereof.

It is to be understood that, throughout the application, the term “transverse” is not intended to be understood as precisely perpendicular, rather it is meant as generally passing across or through. For example, first end portion 20a may extend “transversely” through the longitudinal member at the channel site by extending through the longitudinal member at an angle other than 90 degrees with respect to the longitudinal member at the channel site. Similarly, rod 30 may extend “transversely” through the longitudinal member at an angle other than 90 degrees with respect to the longitudinal member.

The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein can be performed on a living animal or on a non-living simulation, such as on a cadaver, cadaver heart, simulator (e.g., with the body parts, tissue, etc. being simulated), etc.

1. A system for use with tissue of a heart, the system comprising:
a helical member; and
a guide assembly, having a distal part that is transluminally advanceable to the heart while in a delivery state, the guide assembly comprising:
a guide frame, intracardially expandable toward an expanded state,
a guide rail, and
multiple fasteners, configured to hold the guide rail in a guide arrangement around at least part of the guide frame, and
a driver, configured to, while the guide rail is in the guide arrangement, anchor the helical member along an intracardial surface of tissue adjacent the guide frame, guided by the guide rail.

2. The system according to claim 1, wherein the guide frame is self-expanding.

3. The system according to claim 1, wherein the system is configured to facilitate the guide assembly withdrawing the guide rail and the guide frame from the heart while the helical member remains in the heart.

4. The system according to claim 1, wherein the driver is configured to anchor the helical member along the intracardial surface of the tissue by advancing the helical member over and along the guide rail.

5. The system according to claim 1, wherein, in the guide arrangement, the guide rail is held, by the fasteners, in an arc around at least part of the guide frame.

6. The system according to claim 1, wherein the guide rail is radiopaque.

7. The system according to claim 1, wherein each of the fasteners is openable within the heart, to decouple the guide frame from the guide rail.

8. The system according to claim 1, wherein the driver is configured to anchor the helical member along the intracardial surface of the tissue via rotation of the helical member.

9. The system according to claim 1, wherein the helical member has an axial length of 5-12 cm.

10. The system according to claim 1, wherein the helical member has a sharpened tip.

11. The system according to claim 1, further comprising a sheath, the distal part of the guide assembly being transluminally advanceable to the heart while in the delivery state within the sheath.

12. The system according to claim 11, wherein:
in the delivery state, the guide frame is constrained within the sheath, and
the guide frame is configured to automatically self-expand within the heart upon becoming deployed out of the sheath.

13. The system according to claim 1, wherein the system comprises an annuloplasty implant, the helical member being a component of the annuloplasty implant.

14. The system according to claim 13, wherein:
the tissue is tissue of an annulus of a valve of the heart, the annulus circumscribing an orifice of the valve,
the guide assembly is configured to position the guide frame through the orifice such that the guide frame is adjacent the annulus,
the intracardial surface of the tissue adjacent the guide frame is an atrial-facing surface of tissue of the annulus, and
the driver is configured to anchor the helical member along the atrial-facing surface of the tissue of the annulus.

15. The system according to claim 1, wherein the helical member defines multiple turns that circumscribe a central channel of the helical member, and the helical member is anchorable along the intracardial surface of the tissue while the guide rail extends within the central channel.

16. The system according to claim 15, wherein the guide rail is configured to limit a depth of penetration of the helical member into the tissue.

17. The system according to claim 15, further comprising a contraction member extending coaxially through the central channel, and a tensioning tool that is configured to contract the tissue along which the helical member is anchored by axially contracting the helical member by applying tension to the contraction member.

18. The system according to claim 17, wherein the contraction member extends coaxially through the central channel alongside the guide rail.

19. The system according to claim 17, wherein the contraction member extends coaxially through the central channel by extending coaxially through a channel defined by the guide rail.

20. The system according to claim 19, wherein, the guide rail is configured such that, while the helical member remains anchored along the tissue, the guide rail is withdrawable from the helical member by sliding the guide rail proximally out of the central channel, leaving the contraction member exposed within the central channel.

21. The system according to claim 20, further comprising a stopper coupled to a distal end of the contraction member, such that tension applied to the contraction member longitudinally contracts the helical member by the stopper inhibiting sliding of the contraction member through the central channel.

22. The system according to claim 21, wherein the stopper is a first stopper, and wherein the system further comprises a second stopper, configured to lock the tension in the contraction member by being couplable to the contraction member proximally from the helical member.

23. The system according to claim 1, wherein the helical member defines a series of turns, and wherein the driver is configured to anchor the helical member along the intracardial surface of the tissue by screwing the helical member along the intracardial surface of the tissue such that a part each turn becomes embedded in the tissue and another part of each turn becomes disposed outside of the tissue.

24. The system according to claim 23, wherein the helical member has a constant pitch.

25. A system for use with tissue of a heart, the system comprising:
an implant; and
a guide assembly, having a distal part that is transluminally advanceable to the heart while in a delivery state, the guide assembly comprising:
a guide frame, intracardially expandable toward an expanded state,
a guide rail, and
multiple fasteners, configured to hold the guide rail in a guide arrangement around at least part of the guide frame, and
a driver, configured to, while the guide rail is in the guide arrangement, secure the implant along an intracardial surface of tissue adjacent the guide frame, guided by the guide rail.

26. The system according to claim 25, wherein the system is configured to facilitate the guide assembly withdrawing the guide rail and the guide frame from the heart while the implant remains in the heart.
